(19) United States
(12) Patent Application Publication
(10) Pub. No.: US 2005/0081268 A1
(43) Pub. Date: Apr. 14, 2005

(54) Bioluminescent plants and methods of making same

(76) Inventors: Steve A. Kay, San Diego, CA (US); Tina Kuhlmann, San Diego, CA (US); Richard A. Lerner, La Jolla, CA (US)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 10/473,945

(22) PCT Filed: Apr. 8, 2002
(86) PCT No.: PCT/US02/11116

Related U.S. Application Data
(60) Provisional application No. 60/282,094, filed on Apr. 6, 2001.

(51) Int. Cl. A01H 1/00; C12N 15/82; C12N 5/04

(52) U.S. Cl. 800/288; 435/419

(57) ABSTRACT

A genetically modified plant cell containing a heterologous nucleotide sequence encoding a bioluminescent polypeptide, which is expressed in amount sufficient to produce at least about 750,000 photons of visible light/mm²/second, is provided, as is a visibly bioluminescent transgenic plant, which contains such a genetically modified plant cell. Also provided is a recombinant nucleic acid molecule, which contains a plant translational enhancer operatively linked to a nucleotide sequence encoding a bioluminescent polypeptide. In addition, methods of producing a genetically modified plant cell that is visibly bioluminescent introducing a transgene encoding a bioluminescent polypeptide into a plant cell, whereby the bioluminescent polypeptide is expressed at a level that produces at least about 750,000 photons of visible light/mm²/second are provided, as are kits containing such visibly bioluminescent compositions.

### BACKGROUND OF THE INVENTION

### FIELD OF THE INVENTION

The present invention relates generally to plant biology and more specifically to plants that are genetically modified to exhibit bioluminescence, and to compositions and methods for making such genetically modified bioluminescent plants.

### BACKGROUND INFORMATION

Plants and plant products provide the primary sustenance, either directly or indirectly, for all animal life, including humans. For the majority of the world's human population and for many animals, plants and plant products provide the sole source of nutrition. As the world population increases, the best hope to prevent widespread famine is to increase the quantity and improve the quality of food crops.

Throughout history, a continual effort has been made to increase the yield and nutritious value of food crops. For centuries, plants having desirable characteristics such as greater resistance-to drought conditions or increased size of fruit were crossbred and progeny plants exhibiting the desired characteristics were selected and used to produce seed or cuttings for propagation. Using such classical genetic methods, plants having, for example, greater disease resistance, increased yield, and better flavor have been obtained.

More recently, the methods of genetic engineering have been applied to plant biology, thus expediting the generation of plants having desired characteristics. For example, genetically engineered tomato plants have been generated such that the tomatoes do not continue to ripen after they have been picked from the plant. As a result, there is no need to pick unripe tomatoes and hope that they attain an optimum level of ripeness at the time the consumer is ready to eat them. Instead, the tomatoes can be picked at a time when they are near their optimal ripeness, then shipped to consumers for consumption. Genetically engineered plants that are less susceptible to freezing or to damage by biological pests also have been generated. The availability of such plants has been a boon to the agricultural industry, and provides a measure of certainty that sufficient quantities of food will remain available in the future.

Methods of genetic engineering also have impacted the ornamental plant industry. In addition to protecting ornamental plants from environmental stresses or biological pests, genetic engineering has been used to produce ornamental plants having new and interesting characteristics. For example, genetic engineering has been used to generate roses that are blue in color. The ability to generate ornamental plants having new and unusual characteristics provides a means to expand the ornamental plant market and, therefore, the economy in general. Thus, a need exists to identify interesting and unusual traits that can be expressed by a plant and can create a new market in the ornamental plant industry. The present invention satisfies this need and provides additional advantages.

### SUMMARY OF THE INVENTION

The present invention relates to a genetically modified plant cell that exhibits bioluminescence that is visible to the naked eye. Such a genetically modified plant cell contains a heterologous nucleotide sequence that encodes a bioluminescent polypeptide, which, upon expression, can result in the production of at least about 750,000 photons of visible light/mm²/second by the plant cell. The bioluminescent polypeptide can be any polypeptide that is bioluminescent, or can act in concert with a second molecule such that visible light is produced. In one embodiment, the genetically modified plant cell contains a heterologous nucleotide sequence encoding luciferase or a luciferase variant, for example, luc⁺. Upon contact of a genetically modified plant expressing luciferase or a variant thereof with luciferin, a sufficient amount of visible light is produced such that the light is visible to the naked eye. Such a genetically modified plant cell generally expresses at least about 100 pg luc⁺/μg protein as determined by western blot analysis such that, upon contact of the plant cell with luciferin, the cell is visibly bioluminescent.

In another embodiment, the genetically modified plant cell, or the plant cell from which the genetically modified plant cell is derived, is contacted with an agent that reduces or inhibits pigment formation in the plant, for example, carotenoid synthesis, thereby reducing or inhibiting chlorophyll synthesis. Since chlorophyll in a plant cell can absorb photons generated by the luciferase in the genetically modified plant cell, contact of the genetically modified plant cell, or a transgenic plant derived therefrom, with an amount of a carotenoid inhibitor sufficient to reduce or inhibit chlorophyll synthesis provides a means to enhance photon emission from the plant cell and, therefore, enhance the visible bioluminescence.

In still another embodiment, a visibly bioluminescent transgenic plant is generated from the genetically modified plant cell containing a heterologous nucleotide sequence encoding a bioluminescent polypeptide. As such, the invention relates to such a transgenic plant, as well as to a plant cell or tissue obtained from the transgenic plant, a seed produced by the transgenic plant, and a cDNA or genomic DNA library prepared from the transgenic plant or from a plant cell or plant tissue obtained from the transgenic plant. The transgenic plant can be a monocot, or can be a dicot, for example, an angiosperm such as a cereal plant, a leguminous plant, an oilseed plant, a hardwood tree, or an ornamental plant such as a petunia, an orchid, a carnation or the like.

The present invention also relates to a recombinant nucleic acid molecule, which comprises a translational enhancer, which enhances translation in a plant cell, operatively linked to a nucleotide sequence encoding a bioluminescent polypeptide. The translational enhancer can be any translational enhancer, for example, a plant potyvirus translational enhancer such as tobacco etch virus (TEV) translational enhancer, a tobacco mosaic virus omega translational enhancer, or a translational enhancer comprising a Kozak sequence. The encoded bioluminescent polypeptide can be a luciferase polypeptide or variant thereof, for example, the luc⁺ luciferase variant.

A recombinant nucleic acid molecule of the invention also can contain a transcriptional regulatory element, i.e., promoter, enhancer, etc., that enhances transcription in a plant cell, for example, a plant virus enhancer such as a cauliflower mosaic virus (CaMV) 35S enhancer, or a regulatory element from any other organism, for example, an actin 2 regulatory element or a metallothionein regulatory element, which comprises a promoter and, optionally, an enhancer, wherein the transcriptional regulatory element is operatively linked to the plant translational enhancer and nucleotide sequence encoding the bioluminescent polypeptide. In one embodiment, a recombinant nucleic acid molecule contains, in operative linkage, a CaMV 35S enhancer, a CaMV 35S promoter, a TEV translational enhancer, a nucleotide sequence encoding a luciferase polypeptide or variant thereof, and a CaMV 35S terminator. In another embodiment, the CaMV 35S enhancer is a dual CaMV 35S enhancer, and the luciferase polypeptide or variant thereof is the luc⁺ luciferase variant. Such a recombinant nucleic acid molecule is exemplified herein by the nucleotide sequence set forth as nucleotides 8366 to 11,113 of SEQ ID NO:2. In still other embodiments, the transcriptional regulatory element in a recombinant nucleic molecule of the invention is a metallothionein regulatory element or an actin 2 regulatory element, and the translational enhancer is an omega enhancer, for example, in a recombinant nucleic acid molecule having a sequence set forth as nucleotides 8340 to 12,098 of SEQ ID NO:4 or as nucleotides 6 to 3570 of SEQ ID NO:5.

The present invention further relates to a vector containing a recombinant nucleic acid molecule comprising a translational enhancer active in a plant operatively linked to a nucleotide sequence encoding a bioluminescent polypeptide. Preferably, the vector is an expression vector. In one embodiment, the recombinant nucleic acid molecule contained in the vector comprises, in operative linkage, a CAMV 35S enhancer, a CaMV 35S promoter, a TEV translational enhancer, a nucleotide sequence encoding a luciferase polypeptide or variant thereof, and a CaMV 35S terminator. In another embodiment, the recombinant nucleic acid molecule contained in the vector comprises, in operative linkage, a dual CaMV 35S enhancer, a CaMV 35S promoter, a TEV translational enhancer, a nucleotide sequence encoding a luc⁺ luciferase variant, and a CaMV 35S terminator. In still another embodiment, the recombinant nucleic acid molecule contained in the vector comprises the nucleotide sequence set forth as nucleotides 8366 to 11,113 of SEQ ID NO:2. In yet another embodiment, the transcriptional regulatory element in the recombinant nucleic acid molecule comprises an actin 2 promoter or a metallothionein promoter, and the translation enhancer is an omega enhancer, for example, in the recombinant nucleic acid molecules set forth as nucleotides 8340 to 12,098 of SEQ D) NO:4 or as nucleotides 6 to 3570 of SEQ ID NO:5. Vectors of the invention are exemplified herein by those as set forth in SEQ ID NO:1, SEQ ID NO:2, SEQ ID NO:4 and SEQ ID NO:5. The present invention also provides a cell containing a recombinant nucleic acid molecule of the invention, or containing a vector of the invention.

The present invention further relates to a method of producing a genetically modified plant cell that is visibly bioluminescent. A method of the invention can be performed, for example, by introducing a transgene comprising a nucleotide sequence encoding a bioluminescent polypeptide into a plant cell, whereby the bioluminescent polypeptide is expressed at a level that produces at least about 750,000 photons of visible light/mm²/second. The transgene useful in a method of making a genetically modified plant cell that is visibly bioluminescent generally includes a translational enhancer that is active in a plant, for example, a plant translational enhancer such as a plant potyvirus translational enhancer (e.g., a TEV translational enhancer), a tobacco mosaic virus (TMV) translational enhancer such as the omega enhancer, operatively linked to the nucleotide sequence encoding a bioluminescent polypeptide, for example, a luciferase polypeptide or variant thereof. The transgene also can contain a plant virus enhancer, for example, a CaMV 35S enhancer, which is operatively linked to the plant translational enhancer and nucleotide sequence encoding a bioluminescent polypeptide. In one embodiment, a transgene useful in making a visibly luminescent genetically modified plant cell contains, in operative linkage, a dual CaMV 35S enhancer, a CaMV 35S promoter, a TEV translational enhancer, a nucleotide sequence encoding a luciferase polypeptide or variant thereof, and a CaMV 35S terminator. In another embodiment, the nucleotide sequence encodes a luc⁺ luciferase variant polypeptide. Such transgenes are exemplified by the nucleotide sequences set forth as nucleotides 8366 to 11,113 of SEQ ID NO:2, nucleotides 8340 to 12,098 of SEQ ID NO:4, and nucleotides 6 to 3570 of SEQ ID NO:5.

A method of the invention can further include contacting the plant cell, or the genetically modified plant cell derived therefrom, with an agent that inhibits the synthesis of a pigment such as carotenoids, which can absorb light generated by the luciferase. For example, the genetically modified plant cell can be contacted with an agent such as norfluorazon, which reduces or inhibits chlorophyll production in the plants, thus allowing increased emission of photons from the plant cell, or from a trrnsgenic plant derived from the plant cell.

A visibly bioluminescent genetically modified plant cell made according to a method of the invention generally contains the transgene stably maintained in the plant cell genome. Preferably, the transgene is integrated into the plant cell genome. As such, the present invention also relates to a genetically modified plant cell produced by a method of the invention, as well as to a transgenic plant containing or generated from such a genetically modified plant cell.

The present invention also relates to kit, which contains a genetically modified plant cell of the invention, or a derivative of the genetically modified plant cell, for example, a transgenic plant derived from the genetically modified plant cell, or a cell, a tissue, or an organ of such a transgenic plant. As such, a kit of the invention can contain one or more flowers, bracts, leaves or other tissues or organs, which, upon contact with luciferin, are visibly luminescent.

Accordingly, a kit of the invention can further include an amount of luciferin sufficient for generating visible bioluminescence of the genetically modified plant cell or the derivative of the genetically modified plant cell, and also can include a plurality of such amounts of luciferin, thus allowing the generation of visible bioluminesce a number of times, as desired. In addition, a kit of the invention can include an amount of a inhibitor that reduces or inhibits pigment synthesis in the genetically modified plant cell or the derivative of the genetically modified plant cell. For example, the kit can contain a carotenoid synthesis inhibitor such as norfluorazon, which reduces or inhibits chlorophyll production.

In another embodiment, a kit of the invention contains one or more cuttings, seeds, or other portion or derivative of a visibly bioluminescent plant of the invention such that a visibly luminescent transgenic plant can be grown therefrom. As such, the kit also can include reagents for growing a transgenic plant from the cutting or seed, including, for example, a suitable plant food or other nutrient source required for growth of the particular plant. In addition, the kit can include an amount of a carotenoid inhibitor sufficient for reducing or inhibiting chlorophyll production in a trrnsgenic plant grown from the cutting or seed; and/or can include an amount of luciferin sufficient for generating visible bioluminescence of a transgenic plant grown from the cutting or seed.

### BRIEF DESCRIPTION OF THE FIGURES

FIG. 1 shows a map of the pTK1829 plasmid. Restriction endonuclease sites are indicated, as are the first nucleotide positions of the sites, except that the first and last nucleotide positions of the multiple cloning site are indicated. “luc⁺” indicates region encoding variant luciferase polypeptide; “Amp” indicates ampicillin-resistance gene (see, also, SEQ ID NO:1).

FIG. 2 shows a map of the pPZPTK1829 binary vector plasmid. Restriction endonuclease sites and nucleotide positions are indicated as in FIG. 1. “luc⁺” indicates region encoding variant luciferase polypeptide; “Spec” and “Gent-R” indicate the genes conferring resistance to spectinomycin and gentamycin, respectively. “Ori” indicates the plasmid origin of replication. “LB” and “RB” indicate left border and right border, respectively, of the binary vector that allow transfer of the insert into the plant (see, also, SEQ ID NO:2).

FIG. 3 shows a map of the MT-OM-LUC⁺ binary vector plasmid. Restriction endonuclease sites and nucleotide positions are indicated as in FIG. 1. “luc⁺”, “Spec”, “Gent-R”, “Ori”, and “LB” and “RB” are as in FIG. 2. “MT” indicates the metallothionein transcriptional regulatory element; “Ome” indicates the omega translational enhancer; and “E9 3′” indicates the RbcS E9 polyA region (see, also, SEQ ID NO:4).

FIG. 4 shows a map of the ACT-OM-LUC⁺ binary vector plasmid. Restriction endonuclease sites and nucleotide positions are indicated as in FIG. 1. “luc⁺”, “Spec”, “Gent-R”, “ori”, “LB” and “RB” are as in FIG. 2. “Ome” and “E9 3′” are as in FIG. 3; “ACT” indicates the actin 2 transcriptional regulatory element (see, also, SEQ ID NO:5).

### DETAILED DESCRIPTION OF THE INVENTION

The present invention provides genetically modified plant cells that are visibly bioluminescent due to expression of heterologous polypeptide, and visibly bioluminescent transgenic plants comprising or generated from such genetically modified plant cells. As disclosed herein, the transgenic plants of the invention exhibit bioluminescence that is visible to the human eye. Prior to the present disclosure, plant cells could be genetically modified to express heterologous bioluminescent polypeptides such as luciferase, but light gathering or amplifying instrumentation was required to detect the bioluminescence; one could not simply look at plant comprising such cells directly and detect the bioluminescence. It is noted that there is a report on the world wide web, at the URL “hybridorchids.com”, of a bioluminescent orchid. However, no published information for making such orchids is available and the information on the website has not been updated since 2000. The present invention provides compositions and methods that obviate the previous limitations that prevented the generation of visibly detectable plants and plant cells. As disclosed herein, the visibly bioluminescent genetically modified plant cells and transgenic plants of the invention are useful as research tools, and have great ornamental value.

A genetically modified plant cell or transgenic plant of the invention is visibly bioluminescent due to expression in the plant cell, or in plant cells of the transgenic plant, of a bioluminescent polypeptide. As used herein, the term “bioluminescent polypeptide” refers to a polypeptide that can be expressed in a living organism and exhibits or effects the emission of visible light. A bioluminescent polypeptide can inherently emit visible light such as fluorescence, or can be contacted with a physical or chemical agent such that a reaction occurs and visible light is produced. For example, a genetically modified plant cell can contain a heterologous nucleotide sequence encoding luciferase such that, upon expression of the luciferase and contact of the plant cell with luciferin, a chemiluminescent reaction occurs and visible light is emitted. As used herein, the term “visible light” refers to electromagnetic radiation associated with the visible portion of the electromagnetic spectrum, generally electromagnetic radiation having a wavelength between about 100 to 1000 nanometers (nm), and particularly between about 350 to 750 nm.

Luciferase polypeptides and luciferase variants are examples of bioluminescent polypeptides that are particularly useful for practicing the methods and producing the compositions of the invention. As used herein, the term “luciferase variant” refers to a modified luciferase polypeptide that contains one or more amino acid additions, deletions or substitutions as compared to a corresponding wild type luciferase polypeptide, and that, when contacted with an appropriate substrate such as luciferin, undergoes a reaction that results in-the production of visible light.

Luciferases include bacterial luciferases such as Vibrio harveyi or V. fisheri luciferase, marine ostracod crustacean luciferases such a Vargula hilgendorii luciferase, dinoflagellate luciferases such as Gonyaulax ploydra luciferase, coelenterate luciferases such as Renilla renifornis (sea pansy) luciferase, or Coleoptera (beetle) luciferases such as an Elateridae (click beetle) family luciferase, a Phengodidae (glow worm) family luciferase, or a Lampyridae (firefly) luciferase, for example, a Photintus carolinus or P. pyralis luciferase, and the like, including modified forms thereof (see, for example, U.S. Pat. No. 4,968,613; U.S. Pat. No. 5,221,623; U.S. Pat. No. 5,229,285; U.S. Pat. No. 5,330,906; U.S. Pat. No. 5,604,123; U.S. Pat. No. 5,618,722; U.S. Pat. No 5,674,713; U.S. Pat. No. 5,814,465; and U.S. Pat. No. 5,843,746, each of which is incorporated herein by reference). A luciferase variant is exemplified by the firefly luciferase variant, luc⁺ (U.S. Pat. No. 5,670,356; Sherf and Wood, Promega Notes 49:14, 1994, each of which is incorporated herein by reference), which lacks a peroxisome localizing domain, can be particularly useful for preparing a genetically modified plant cell or visibly bioluminescent transgenic plant of the invention. Polynucleotide sequences encoding luciferases and mutant luciferases are well known (see above references) and vectors containing such polynucleotides are commercially available (Promega; Madison Wis.).

As used herein, the term “visibly bioluminescent” refers to the emission of a sufficient amount of visible light such that it can be seen by a human eye. As such, the use of light gathering, light amplifying, light enhancing or light accumulating instrumentation is not required to detect the bioluminescence, although such instrumentation can be used, for example, to quantitate the amount of visible light emitted. It should be recognized that a visibly bioluminescent plant cell may not be visible to the unaided eye due to its small size, but can be seen using a magnifying glass, microscope, or the like. Such a plant cell is considered to be visibly bioluminescent for purposes of the present invention because light gathering, accumulating or amplifying instrumentation such as a luminometer, photomultiplier, image enhancing video system, spectrophotometer, film emulsion, digitizing equipment, or the like is not required.

The light produced by a visibly bioluminescent transgenic plant can be seen by a person or other mammal looking at the plant, particularly when the plant is in a dark environment, for example, in a darkened room or outside at night. In addition, a bioluminescent plant of the invention can be photographed using a camera, including any generally used camera such as a 35 mm single lens reflex camera, a digital camera, a Polaroid® camera, and the like, such that a picture of the glowing plant can be obtained. As disclosed herein, a genetically modified plant cell, or a plant, plant tissue or plant organ comprising the plant cell that emits at least about 750,00 photons of visible light/mm²second, generally at least about 850,000 photons of visible light/mm²/second, and particularly at least about 1×10⁶ (one million) photons of visible light/mm²/second is visibly bioluminescent (see Example 2C, Table 4).

As disclosed herein, contact of a genetically modified plant cell of the invention, or of a plant cell from which the genetically modified plant cell is derived, or of a transgenic plant of the invention with an agent that reduces or inhibits the synthesis of one or more pigments in the plant or plant cell. By reducing the amount of pigmentation of a plant, or a portion of the plant such as the leaves or flowers, absorption of bioluminescence by the pigment is reduced, thereby enhancing the emission of photons from the plant and, therefore, the visible bioluminescence. For example, contact of the genetically modified plant cell or a transgenic plant of the invention with an inhibitor of carotenoid synthesis can reduce or inhibit chlorophyll synthesis in the plant, thus reducing absorption of photons by chlorophyll in the plant and allowing greater emission of the photons from the plant. Inhibitors of carotenoid synthesis in plants are well known and include herbicides such as norfluorazone, which is commercially available as ZORIAL® herbicide (Syngenta Crop Production; Greensboro N.C.).

In another embodiment, a visibly bioluminescent transgenic plant is generated from the genetically modified plant cell containing a heterologous nucleotide sequence encoding a bioluminescent polypeptide. As such, the invention relates to such a transgenic plant, as well as to a plant cell or tissue obtained from the transgenic plant, a seed produced by the transgenic plant, and a cDNA or genomic DNA library prepared from the trrnsgenic plant or from a plant cell or plant tissue obtained from the transgenic plant. The transgenic plant can be a monocot, or can be a dicot, for example, an angiosperm such as a cereal plant, a leguminous plant, an oilseed plant, a hardwood tree, or an ornamental plant such as a petunia, a carnation or the like. In one embodiment, plants useful for purposes of the present invention include orchids. In another embodiment, plants useful for purposes of the present invention exclude orchids.

The present invention provides a recombinant nucleic acid molecule, which includes a heterologous nucleotide sequence encoding a bioluminescent polypeptide operatively linked to one or more transcriptional or translational regulatory elements, or a combination thereof. As used herein, the term “heterologous” is used in a comparative sense with respect to a nucleotide sequence encoding a bioluminescent polypeptide to indicate either that the nucleotide sequence is not an endogenous nucleotide sequence in a plant cell into which it is to be introduced, or that the nucleotide sequence is part of a construct such that it is in a form other than it normally would be found in a plant cell in nature. For example, a heterologous nucleotide sequence can encode a firefly luciferase, which is not normally expressed in a plant cell and, therefore, is heterologous with respect to the plant cell.

The regulatory element or combination of regulatory elements is selected such that the bioluminescent polypeptide can be expressed in a plant cell at a level sufficient for the plant cell, or a plant comprising the cell, to visibly bioluminesce. The term “regulatory element” is used broadly herein to refer to a nucleotide sequence that, when operatively linked to a second, expressible nucleotide effects transcription or translation of the nucleotide sequence such that a ribonucleic acid (RNA) molecule or polypeptide, respectively, can be generated, or encodes a domain that confers a desirable characteristic on a polypeptide containing the domain. For purposes of the present invention, a regulatory element generally increases the amount of transcription or translation of an operatively linked nucleotide sequence encoding the bioluminescent polypeptide, or provides a means to localize a bioluminescent polypeptide to a particular location in a cell, particularly a plant cell. Transcriptional and translational regulatory elements are well known and include promoters, enhancers, 3′-untranslated or 5′-untranslated sequences of transcribed sequence, for example, a poly-A signal sequence or other transcription termination signal, or other protein or RNA stabilizing elements, or other gene expression control elements known to regulate gene expression or the amount of expression of a gene product. A regulatory element useful for constructing a recombinant nucleic acid molecule of the invention can be isolated from a naturally occurring genomic DNA sequence or can be synthetic, for example, a synthetic promoter.

Transcriptional regulatory elements can be constitutively expressed regulatory elements, which maintain gene expression at a relatively constant level of activity, or can be inducible regulatory elements. Constitutively expressed regulatory elements can be expressed in any cell type, for example an actin promoter such as an actin 2 promoter, or an elongation factor (EF) promoter such as an EF1α promoter; or can be a tissue specific regulatory element, which is expressed only in one or a few specific cell types, a phase specific regulatory element, which is expressed only during particular developmental or growth stages of a plant cell, or the like. A regulatory element such as a tissue specific or phase specific regulatory element or an inducible regulatory element useful in constructing a recombinant nucleic acid molecule of the invention can be a regulatory element that generally, in nature, is found in a plant genome. However, the regulatory element also can be from an organism other than a plant, including, for example, from a plant virus, an animal virus, or a cell from an animal or other multicellular organism.

As used herein, the term “tissue specific or phase specific regulatory element” means a nucleotide sequence that effects transcription in only one or a few cell types, or only during one or a few stages of the life cycle of a plant, for example, only for a period of time during a particular stage of growth, development or differentiation. The terms “tissue specific” and “phase specific” are used together herein in referring to such a regulatory element because a single regulatory element can have characteristics of both types of regulatory elements. For example, a regulatory element active only during a particular stage of plant development also can be expressed only in one or a few types of cells in the plant during the particular stage of development. A tissue specific or phase specific regulatory element can be useful for producing a genetically modified plant that is bioluminescent only in particular organs or tissues, or only at a particular developmental stage.

Numerous tissue specific or phase specific regulatory elements have been described and are known and available to those in the art. Such tissue or phase specific regulatory elements include, for example, the AGL8/FRUITFULL regulatory element, which is activated upon floral induction (Hempel et al., Development 124:3845-3853, 1997, which is incorporated herein by reference); root specific regulatory elements such as the regulatory elements from the RCP1 gene and the LRP1 gene (Tsugeki and Fedoroff, Proc. Natl. Acad. Sci., USA 96:12941-12946, 1999; Smith and Fedoroff, Plant Cell 7:735-745, 1995, each of which is incorporated herein by reference); flower specific regulatory elements such as the regulatory elements from the LEAFY gene and the APETELA1 gene (Blazquez et al., Development 124:3835-3844, 1997, which is incorporated herein by reference; Hempel et al., supra, 1997); seed specific regulatory elements such as the regulatory element from the oleosin gene (Plant et al., Plant Mol. Biol. 25:193-205, 1994, which is incorporated herein by reference), and dehiscence zone specific regulatory element. Additional tissue specific or phase specific regulatory elements include the Zn13 promoter, which is a pollen specific promoter (Hamilton et al., Plant Mol. Biol. 18:211-218, 1992, which is incorporated herein by reference); the UNUSUAL FLORAL ORGANS (UFO) promoter, which is active in apical shoot meristem; the promoter active in shoot meristems (Atanassova et al., Plant J. 2:291, 1992, which is incorporated herein by reference), the cdc2a promoter and cyc07 promoter (see, for example, Ito et al., Plant Mol. Biol. 24:863, 1994; Martinez et al., Proc. Natl. Acad. Sci., USA 89:7360, 1992; Medford et al., Plant Cell 3:359, 1991; Terada et al., Plant J. 3:241, 1993; Wissenbach et al., Plant J. 4:411, 1993, each of which is incorporated herein by reference); the promoter of the APETELA3 gene, which is active in floral meristems (Jack et al., Cell 76:703, 1994, which is incorporated herein by reference; Hempel et al., supra, 1997); a promoter of an agamous-like (AGL) family member, for example, AGL8, which is active in shoot meristem upon the transition to flowering (Hempel et al., supra, 1997); floral abscission zone promoters; L1-specific promoters; and the like. Such tissue specific or phase specific regulatory elements can be used to construct a recombinant nucleic acid useful for producing, for example, a transgenic plant that is visibly bioluminescent in one or a few selected tissues or organs such as in flowers or in leaves, or that are expressed only during a particular stage of development such as during flowering.

Inducible regulatory elements also are useful for constructing a recombinant nucleic acid molecule of the invention. As used herein, the term “inducible regulatory element” means a regulatory element that, when exposed to an inducing agent, effects an increased level of transcription of an operatively linked nucleotide sequence encoding the bioluminescent polypeptide as compared to the level of transcription, if any, in the absence of an inducing agent. Inducible regulatory elements can be those that have no basal or constitutive activity and only effect transcription upon exposure to an inducing agent, or those that effect a basal or constitutive level of transcription, which is increased upon exposure to an inducing agent. Inducible regulatory elements that effect a basal or constitutive level of expression generally are particularly useful where the induced level of transcription is substantially greater than the basal or constitutive level of expression, for example, at least about two-fold greater, or at least about five-fold greater. Particularly useful inducible regulatory elements do not have a basal or constitutive activity, or increase the level of transcription at least about ten-fold greater than a basal or constitutive level of transcription associated with the regulatory element.

The term “inducing agent” is used to refer to a chemical, biological or physical agent that effects transcription from an inducible regulatory element. In response to exposure to an inducing agent, transcription from the inducible regulatory element generally is initiated de novo or is increased above a basal or constitutive level of expression. Such induction can be identified using the methods disclosed herein, including detecting an increased level of mRNA encoding the bioluminescent polypeptide. The use of an inducible regulatory element in a recombinant nucleic acid molecule of the invention provides a means to express the bioluminescent polypeptide only at a desired time, thus preventing extraneous transcriptional or translational activity in the plant cell.

An inducing agent useful in a method of the invention is selected based on the particular inducible regulatory element. For example, the inducible regulatory element can be a metallothionein (MT) regulatory element such as an MT2B regulatory element, a copper inducible regulatory element, or a tetracycline inducible regulatory element, the transcription from which can be effected in response to various metal ions, to copper or to tetracycline, respectively (Furst et al., Cell 55:705-717, 1988; Mett et al., Proc. Natl. Acad. Sci., USA 90:4567-4571, 1993; Gatz et al., Plant J. 2:397-404, 1992; Roder et al., Mol. Gen. Genet. 243:32-38, 1994, each of which is incorporated herein by reference). The inducible regulatory element also can be an ecdysone regulatory element or a glucocorticoid regulatory element, the transcription from which can be effected in response to ecdysone or other steroid (Christopherson et al., Proc. Natl. Acad. Sci., USA 89:6314-6318, 1992; Schena et al., Proc. Natl. Acad. Sci., USA 88:10421-10425, 1991, each of which is incorporated herein by reference). In addition, the regulatory element can be a cold responsive regulatory element or a heat shock regulatory element, the transcription of which can be effected in response to exposure to cold or heat, respectively (Takahashi et al., Plant Physiol. 99:383-390, 1992, which is incorporated herein by reference). Additional regulatory elements useful in the methods or compositions of the invention include, for example, the spinach nitrite reductase gene regulatory element (Back et al., Plant Mol. Biol. 17:9, 1991, which is incorporated herein by reference); a light inducible regulatory element (Feinbaum et al., Mol. Gen. Genet. 226:449, 1991; Lam and Chua, Science 248:471, 1990, each of which is incorporated herein by reference), a plant hormone inducible regulatory element (Yamaguchi-Shinozaki et al., Plant Mol. Biol. 15:905 (1990); Kares et al., Plant Mol. Biol. 15:225 (1990), each of which is incorporated herein by reference), and the like. A circadian rhythm regulatory element such as a cab2 promoter (see, for example, Millar et al., Plant Cell 4:1075-1087, 1992, which is incorporated herein by reference) can also be considered an example of an inducible regulatory element, since expression from such a promoter appears to occur in response to a factor associated with a time of day.

A regulatory element operatively linked to a nucleotide sequence encoding a bioluminescent polypeptide also can comprise an enhancer, including a transcriptional enhancer, translational enhancer, or both. A transcriptional enhancer useful for purposes of the present invention can be any enhancer generally associated with a plant gene or an enhancer from another organism that has enhancer activity in a plant cell, for example, a plant virus enhancer, or an enhancer from a gene expressed in an invertebrate cell, or a vertebrate cell such as a mammalian cell, or an enhancer of a virus that infects such cells. A cauliflower mosaic virus (CaMV) 35S enhancer is an example of a plant virus enhancer that is useful for increasing the transcriptional activity of a nucleotide sequence to which it is operatively linked. Thus, in one embodiment, a recombinant nucleic acid molecule of the invention-contains a CaMV 35S enhancer operatively linked to a nucleotide sequence encoding the bioluminescent polypeptide. Preferably, the CaMV 35S enhancer is a dual enhancer, comprising two copies of the CaMV 35S enhancer nucleotide sequence. An actin 2 regulatory element provides another example of a transcriptional regulatory element, comprising a promoter and an enhancer, that is useful in constructing a composition of the invention or practicing a method of the invention (see nucleotides 6 to 1145 of SEQ ID NO:5). A metallothionein regulatory element provides another example of a transcriptional regulatory element useful for purposes of the present invention, and provides the additional advantage that it includes an inducible enhancer (see nucleotides 11,000 to 12,098 of SEQ ID NO:4).

A translational enhancer, which enhances translation of a polypeptide in a plant cell (also referred to generally herein as a plant translational enhancer), useful in a recombinant nucleic acid molecule of the invention similarly can be any translational enhancer that generally is associated with a plant gene or with a gene of another organism. Examples of a plant translational enhancer include a plant potyvirus translational enhancer such as the tobacco etch virus (TEV) translational enhancer, and a tobacco mosaic virus translational enhancer such as the omega enhancer (see, for example, nucleotides 1169 to 1235 of SEQ ID NO:5; see, also, Carrington and Freed, J. Virol. 64:1590, 1990; Gallie et al., Plant Cell 1:301-311, 1989, each of which is incorporated herein by reference), as well as the nucleotide sequence beginning about 101 nucleotide upstream of the potato virus S (PSV) coat protein gene (Turner and Foster, Arch. Virol. 142:167-175, 1997, which is incorporated herein by reference). A recombinant nucleic acid molecule of the invention can also contain regulatory elements required for termination of transcription, for example, a polyadenylation signal. A CaMV 35S terminator is an example of a transcription termination signal useful in constructing a recombinant nucleic acid molecule.

A recombinant nucleic acid molecule of the invention also can contain regulatory elements required for initiation or termination of translation or both, including, for example, an internal ribosome entry site (IRES), a Kozak sequence, a nucleotide sequence encoding an initiator methionine residue, and a nucleotide sequence encoding a STOP codon, which terminates translation. The regulatory element also can be a nucleotide sequence encoding a leader sequence, signal peptide, cell compartmentalization domain, or the like, and can be included in the construct, for example, where it is desired to localize the bioluminescent polypeptide to a particular compartment such as the cytosol, nucleus, a chloroplast, or another subcellular organelle in a plant cell. Such intracellular localization can be particularly useful where the genetically modified cell, or plant containing the cell, is to be used as a research tool, for example, a particular intracellular compartment of a plant cell.

A translation initiation or termination element or other regulatory element can be contained in the nucleotide sequence encoding the bioluminescent polypeptide can be a regulatory element that is normally associated with the nucleotide sequence in nature and is a part of the nucleotide sequence as a consequence of its isolation from a natural source, or can be heterologous to the nucleotide sequence and operatively linked thereto. Additional regulatory elements such as an intron sequence, for example, from Adh1 or bronzel, or a viral leader sequence, for example, from TMV, MCMV or AIVIV, also enhance expression of a nucleotide sequence encoding a bioluminescent polypeptide and, therefore, can be a component of a recombinant nucleic acid molecule of the invention.

As disclosed herein, combinations of regulatory elements can be selected such that a recombinant nucleic acid molecule comprising a nucleotide sequence encoding a bioluminescent polypeptide can be constructed, and wherein the recombinant nucleic acid molecule allows for expression of the bioluminescent polypeptide in a plant cell such that the plant cell is visibly bioluminescent. A recombinant nucleic acid molecule of the invention is exemplified by, in operative linkage, a CaMV 35S enhancer, a CaMV 35S promoter, a TEV translational enhancer, a nucleotide sequence encoding a luciferase polypeptide or variant thereof, and a CaMV 35S terminator. In one embodiment, the CaMV 35S enhancer comprises a dual CaMV 35S enhancer, and the luciferase polypeptide or variant thereof is a luc+luciferase variant. In another embodiment, a recombinant nucleic acid molecule of the invention is exemplified by the nucleotide sequence set forth as about nucleotides 8366 to 11,113 of SEQ ID NO:2.

A recombinant nucleic acid molecule of the invention also is exemplified by the sequence set forth as nucleotides 8340 to 12,098 of SEQ ID NO:4, which includes, in operative linkage, a metallothionein gene (MT2B) regulatory element comprising an enhancer and promoter (nucleotides 11,000 to 12,098 of SEQ ID NO:4), an omega translational enhancer (nucleotides 10,675 to 10,741 of SEQ ID NO:4), a nucleotide sequence encoding a luciferase polyp eptide or variant thereof such as luc⁺ (nucleotides 9015 to 10,667 or SEQ ID NO:4) and an RbcS E9 polyA region (nucleotides 8340 to 8981 of SEQ ID NO:4). In addition, a recombinant nucleic acid molecule of the invention also is exemplified by the sequence set forth as nucleotides 6 to 3570 of SEQ ID NO:5, which includes, in operative linkage, an actin 2 regulatory element comprising an enhancer and promoter (nucleotides 6 to 1145 of SEQ ID NO:5), an omega translational enhancer (nucleotides 1169 to 1235 of SEQ ID NO:5), a nucleotide sequence encoding a luciferase polypeptide or variant thereof such as luc⁺ (nucleotides 1243 to 2895 of SEQ ID NO:5), and an RbcS E9 polyA region (nucleotides 2929 to 3570 of SEQ ID NO:5).

A recombinant nucleic acid molecule of the invention comprises a nucleotide sequence encoding a bioluminescent polypeptide operatively linked to one or more regulatory elements, for example, a translational enhancer. As used herein, the term “operatively linked,” when used in reference to a regulatory element and a second nucleotide sequence, which can be another regulatory element or a coding or other sequence, means that the regulatory element is positioned with respect to the second nucleotide sequence such that the regulatory element effects its function with respect to the second nucleotide sequence in substantially the same manner as it does when the regulatory element is present in its natural position in a genome. Transcriptional promoters, for example, generally act in a position and orientation dependent manner, and usually are positioned at or within about five nucleotides to about fifty nucleotides 5′ (upstream) of the start site of transcription of a gene in nature. In comparison, enhancers can act in a relatively position or orientation independent manner, and can be positioned several hundred or thousand nucleotides upstream or downstream from a transcription start site, or in an intron within the coding region of a gene, yet still be operatively linked to the coding region so as to enhance transcription. The relative positions and orientations of various regulatory elements, including the positioning of a transcribed regulatory sequence such as an IRES or a translated regulatory element such as a cell compartmentalization domain in an appropriate reading frame, are well known and methods for operatively linking such elements are routine in the art (see, for example, Sambrook et al., “Molecular Cloning: A laboratory manual” (Cold Spring Harbor Laboratory Press 1989); Ausubel et al., “Current Protocols in Molecular Biology” (John Wiley and Sons, Baltimore Md. 1987, and supplements through 1995), each of which is incorporated herein by reference).

As used herein, the term “nucleic acid molecule” or “polynucleotide” or “nucleotide sequence” means a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms are used herein to include naturally occurring nucleic acid molecules, which can be isolated from a cell, as well as synthetic molecules, which can be prepared, for example, by methods of chemical synthesis or by enzymatic methods such as by the polymerase chain reaction (PCR). The term “recombinant” is used herein to refer to a nucleic acid molecule that is produced by linking together at least two nucleotide sequences that are not generally linked together in nature. As such, a recombinant nucleic acid molecule encompassed within the present invention is distinguishable from a nucleic acid molecule that may be produced, for example, during meiosis.

In general, the nucleotides comprising a recombinant nucleic acid molecule, polynucleotide or nucleotide sequence are naturally occurring deoxyribonucleotides, such as adenine, cytosine, guanine or thymine linked to 2′-deoxyribose, or ribonucleotides such as adenine, cytosine, guanine or uracil linked to ribose. However, a nucleic acid molecule or nucleotide sequence also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234, 1994; Jellinek et al., Biochemistry 34:11363-11372, 1995; Pagratis et al., Nature Biotechnol. 15:68-73, 1997, each of which is incorporated herein by reference).

Similarly, the covalent bond linking the nucleotides of a nucleotide sequence generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986, 1994; Ecker and Crooke, BioTechnology 13:351360, 1995, each of which is incorporated herein by reference). The incorporation of non-naturally occurring nucleotide analogs or bonds linking the nucleotides or analogs can be particularly useful where the nucleic acid molecule is to be exposed to an environment that can contain a nucleolytic activity, including, for example, a plant tissue culture medium or in a plant cell since the modified molecules can be less susceptible to degradation.

A nucleotide sequence containing naturally occurring nucleotides and phosphodiester bonds, can be chemically synthesized or can be produced using recombinant DNA methods, using an appropriate polynucleotide as a template. In comparison, a nucleotide sequence containing nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995).

A recombinant nucleic acid molecule of the invention can be introduced into a cell as a naked DNA molecule, can be incorporated in a matrix such as a liposome or a particle such as a viral particle, or can be incorporated into a vector. Incorporation of the polynucleotide into a vector can facilitate manipulation of the polynucleotide, or introduction of the polynucleotide into a plant cell. Accordingly, the present invention also provides a vector containing a recombinant nucleic acid comprising a nucleotide sequence encoding a bioluminescent polypeptide operatively linked to one or more regulatory elements.

A vector can be derived from a plasmid or a viral vector such as a T-DNA vector (Horsch et al., Science 227:1229-1231 (1985), which is incorporated herein by reference). If desired, the vector can comprise components of a plant transposable element, for example, a Ds transposon (Bancroft and Dean, Genetics 134:1221-1229, 1993, which is incorporated herein by reference) or an Spm transposon (Aarts et al., Mol. Gen. Genet. 247:555-564, 1995, which is incorporated herein by reference). In addition to containing a recombinant nucleic acid molecule of the invention, the vector also can contain various nucleotide sequences that facilitate, for example, rescue of the vector from a transformed plant cell; passage of the vector in a host cell, which can be a plant, animal, bacterial, or insect host cell; or expression of an encoding nucleotide sequence in the vector, including all or a portion of a rescued coding region. As such, a vector can contain any of a number of additional transcription and translation elements, including constitutive and inducible promoters, enhancers, and the like (see, for example, Bitter et al., Meth. Enzymol. 153:516-544, 1987). For example, a vector can contain elements useful for passage, growth or expression in a bacterial system, including a bacterial origin of replication; a promoter, which can be an inducible promoter; and the like. A vector also can contain one or more restriction endonuclease recognition and cleavage sites, including, for example, a polylinker sequence, to facilitate insertion or removal of a recombinant nucleic acid molecule.

In addition to a nucleotide sequence encoding a bioluminescent polypeptide, a recombinant nucleic acid molecule of the invention, or a vector containing such a recombinant nucleic acid molecule, can contain one or more other expressible nucleotide sequences encoding an RNA or a polypeptide of interest. For example, the additional nucleotide sequence can encode an antisense nucleic acid molecule; an enzyme such as β-galactosidase, β-glucuronidase, luciferase, alkane phosphatase, glutathione S-transferase, chloramphenicol acetyltransferase, guanine xanthine phosphoribosyltransferase, and neomycin phosphotransferase; a viral polypeptide or a peptide portion thereof; or a growth factor or a hormone, which can be a plant growth factor or hormone. Expression of such a nucleotide sequence can provide a means for selecting for a cell containing the construct, for example, by conferring a desirable phenotype to a plant cell containing the nucleotide sequence. For example, the additional nucleotide sequence can be, or encode, a selectable marker, which, when present or expressed in a plant cell, provides a means to identify the plant cell containing the marker. A selectable marker provides a means for screening a population of plants, or plant cells, to identify those having the marker and, therefore, a recombinant nucleic acid molecule comprising a nucleotide sequence encoding a bioluminescent polypeptide. A selectable marker generally confers a selective advantage to the plant cell, or a plant containing the cell, for example, the ability to grow in the presence of a negative selective agent such as an antibiotic or herbicide. A selective advantage also can be due, for example, to an enhanced or novel capacity to utilize an added compound as a nutrient, growth factor or energy source. A selective advantage can be conferred by a single polynucleotide, or its expression product, or by a combination of polynucleotides whose expression in a plant cell gives the cell a positive selective advantage, a negative selective advantage, or both. It should be recognized that expression of a bioluminescent polypeptide also provides a means to a select plant cell containing the encoding nucleotide sequence. However, the use of an additional selectable marker, which, for example, allows a plant cell to survive under otherwise toxic conditions, provides a means to enrich for transformed plant cells containing the recombinant nucleic acid molecule.

Examples of selectable markers include those described above, as well as those that confer antimetabolite resistance, for example, dihydrofolate reductase, which confers resistance to methotrexate (Reiss, Plant Physiol. (Life Sci. Adv.) 13:143-149, 1994); neomycin phosphotransferase, which confers resistance to the aminoglycosides neomycin, kanamycin and paromycin (Herrera-Estrella, EMBO J. 2:987-995, 1983) and hygro, which confers resistance to hygromycin (Marsh, Gene 32:481-485, 1984), trpB, which allows cells to utilize indole in place of tryptophan; hisD, which allows cells to utilize histinol in place of histidine (Hartman, Proc. Natl. Acad. Sci., USA 85:8047, 1988); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, 1987, In: Current Communications in Molecular Biology, Cold Spring Harbor Laboratory ed.); and deaminase from Aspergillus terreus, which confers resistance to Blasticidin S (Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (White et al., Nucl. Acids Res. 18:1062, 1990; Spencer et al., Theor. Appl. Genet. 79:625-631, 1990), a mutant EPSPV-synthase, which confers glyphosate resistance (Hinchee et al., BioTechnology 91:915-922, 1998), a mutant acetolactate synthase, which confers imidazolione or sulfonylurea resistance (Lee et al., EMBO J. 7:1241-1248, 1988), a mutant psbA, which confers resistance to atrazine (Smeda et al., Plant Physiol. 103:911-917, 1993), or a mutant protoporphyrinogen oxidase (see U.S. Pat. No. 5,767,373), or other markers conferring resistance to an herbicide such as glufosinate. In addition, markers that facilitate identification of a plant cell containing the polynucleotide encoding the marker include, for example, luciferase (Giacomin, Plant Sci. 116:59-72, 1996; Scikantha, J. Bacteriol. 178:121, 1996), green fluorescent protein (Gerdes, FEBS Lett. 389:4447, 1996) or fl-glucuronidase (Jefferson, EMBO J. 6:3901-3907, 1997), and numerous others as disclosed herein or otherwise known in the art. Such markers also can be used as reporter molecules.

As disclosed herein, a vector of the invention contains a recombinant nucleic acid molecule comprising one or more regulatory elements in operative linkage with a nucleotide sequence encoding a bioluminescent polypeptide. For example, the recombinant nucleic acid molecule in a vector can contain, in operative linkage, a plant enhancer, plant promoter, a translational enhancer, a nucleotide sequence encoding a bioluminescent polypeptide, a translation termination sequence, and transcription termnination sequence. It should be recognized that one or more of the regulatory elements initially can be a component of the vector or of the recombinant nucleic acid molecule.

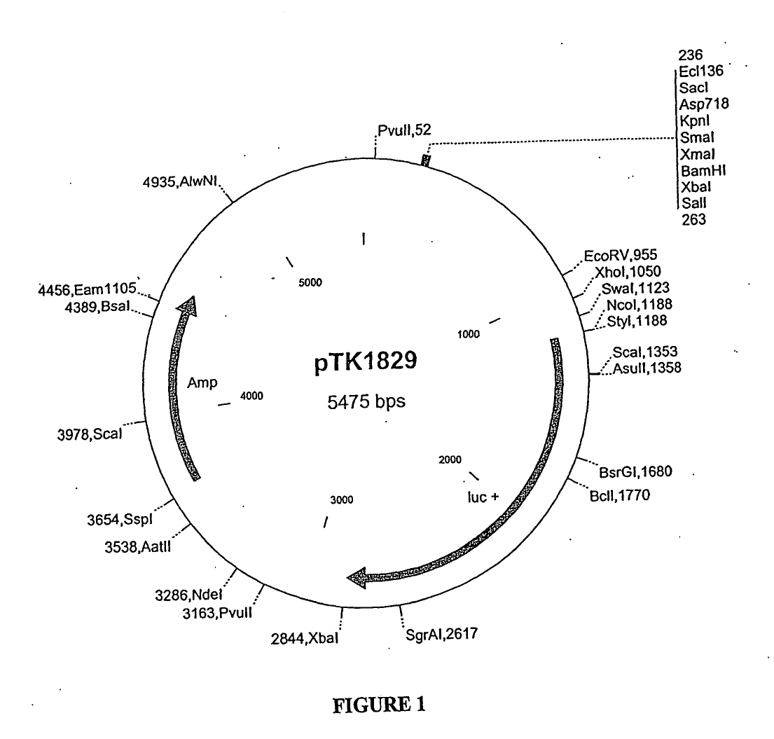

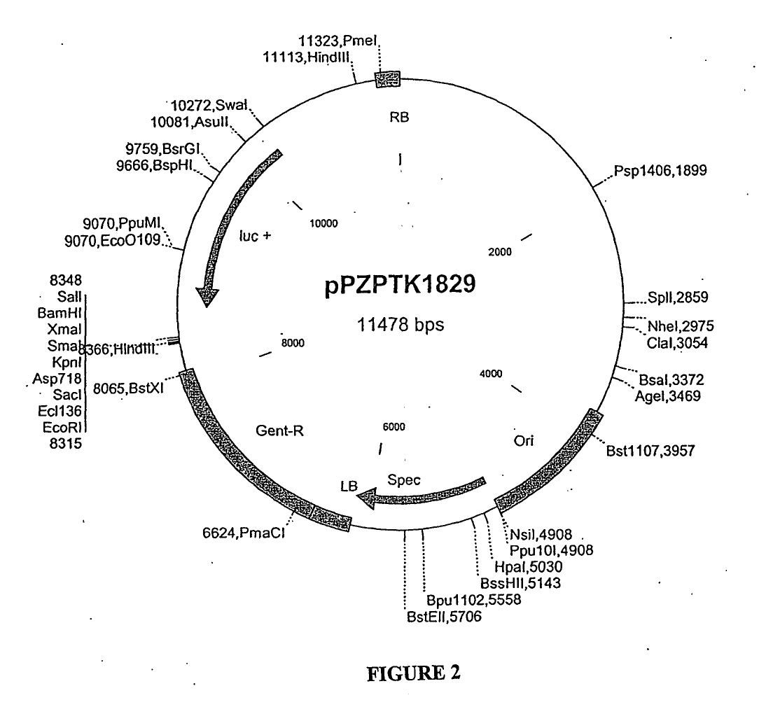

In one embodiment, a vector of the invention contains a recombinant nucleic acid molecule comprising, in operative linkage, a CaMV 35S enhancer, a CaMV 35S promoter, a TEV translational enhancer, a nucleotide sequence encoding a luciferase polypeptide or variant thereof, and a CaMV 35S terminator. In another embodiment, the recombinant nucleic acid molecule contained in the vector comprises, in operative linkage, a dual CaMV 35S enhancer, a CaMV 35S promoter, a TEV translational enhancer, a nucleotide sequence encoding a luc⁺ luciferase variant, and a CaMV 35S terminator. Vectors of the invention are exemplified herein by a vector having the nucleotide sequence of SEQ ID NO:1 (see, also, FIG. 1), and by a vector having the nucleotide sequence of SEQ ID NO:2 (see, also, FIG. 2). Additional vectors of the invention are exemplified by those set forth as SEQ ID NO:4 and SEQ ID NO:5. The present invention also provides a host cell containing a recombinant nucleic acid molecule of the invention, or containing a vector of the invention.

The present invention further provides a method of producing a genetically modified plant cell that is visibly bioluminescent. As used herein, the term “genetically modified,” when used in reference to a plant cell, means that the plant cell contains an exogenously introduced nucleic acid molecule. For purposes of the present invention, the introduced nucleotide sequence generally is a recombinant nucleic acid molecule comprising one or more regulatory elements operatively linked to a nucleotide sequence encoding a bioluminescent polypeptide, or a vector containing such a recombinant nucleic acid molecule. The nucleic acid molecule can be transiently associated with the plant cell, but generally is stably associated with the genetically modified cell, as well as its progeny, either as an autonomously replicating molecule or due to integration into the plant cell genomic DNA or plastid DNA that is stably maintained in the plant cell. If desired, a transgene comprising a recombinant nucleic acid molecule of the invention can be introduced into a plant genome in a site-specific matter, for example, by homologous recombination.

A method of the invention can be performed, for example, by introducing a transgene comprising a recombinant nucleic acid molecule of the invention into a plant cell such that the encoded bioluminescent polypeptide is expressed in the plant cell at a level that can produce at least about 750,000 to 1×10⁶ photons of visible light/mm²/second. As such, the present invention also provides a genetically modified plant cell produced by a method of the invention, as well as to a transgenic plant containing or generated from such a genetically modified plant cell, and to plant tissues, plant cells, plant organs and seeds produced by such a transgenic plant; as well as to a cDNA or genomic DNA library prepared from the transgenic plant, or from a plant cell or plant tissue obtained from the transgenic plant. A method of the invention can further include contacting the plant cell, or the genetically modified plant cell derived therefrom, with an agent that reduces or inhibits pigment synthesis in the plant cell or in a tissue or organ of a transgenic plant of the invention, for example, the synthesis of an anthocyanin, which is a water-soluble red to blue plant pigment; a carotene, which is an orange-yellow pigment located in the chloroplasts; chlorophyll, which is the green pigment in green plants; xanthophyll, which is a yellow to colorless photosynthetic plant pigment, or any other pigment that can absorbs visible light, for example, lycopene, which is a red pigment found in tomatoes. The agent that is selected for reducing or inhibiting pigmentation of a plant is based on the particular pigment, and that the pathway involved in synthesis of the pigment. For example, an agent such as norfluorazon, which inhibits carotenoid synthesis, can be used to reduce the amount of chlorophyll in the plant cell, thus enhancing the emission of a greater number of photons from the plant cell, or from a transgenic plant derived from the plant cell.

The term “plant” is used broadly herein to include any plant at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or aggregate of cells such as a friable callus, or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts usefull for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like.

A transgenic plant can be regenerated from a genetically modified plant cell, i.e., a whole plant can be regenerated from a plant cell; a group of plant cells; a protoplast; a seed; or a piece of a plant such as a leaf, a cotyledon or a cutting. Regeneration from protoplasts varies from species to species of plants. For example, a suspension of protoplasts can be made and, in certain species, embryo formation can be induced from the protoplast suspension, to the stage of ripening and germination. The culture media generally contains various components necessary for growth and regeneration, including, for example, hormones such as auxins and cytokinins; and amino acids such as glutamic acid and proline, depending on the particular plant species. Efficient regeneration will depend, in part, on the medium, the genotype, and the history of the culture. If these variables are controlled, however, regeneration is reproducible.

Regeneration can occur from plant callus, explants, organs or plant parts. Transformation can be performed in the context of organ or plant part regeneration. (see Meth. Enzymol. Vol. 118; Klee et al. Ann. Rev. Plant Physiol. 38:467 (1987), which is incorporated herein by reference). Utilizing the leaf disk-transformation-regeneration method, for example, disks are cultured on selective media, followed by shoot formation in about two to four weeks (see Horsch et al., supra, 1985). Shoots that develop are excised from calli and transplanted to appropriate root-inducing selective medium. Rooted plantlets are transplanted to soil as soon as possible after roots appear. The plantlets can be repotted as required, until reaching maturity.

In vegetatively propagated crops, the mature tralsgenic plants are propagated utilizing cuttings or tissue culture techniques to produce multiple identical plants. Selection of desirable transgenotes is made and new varieties are obtained and propagated vegetatively for commercial use. In seed propagated crops, the mature transgenic plants can be self-pollinated to produce a homozygous inbred plant. The resulting inbred plant produces seeds that contain the introduced transgene, which can comprise a heterologous nucleotide sequence encoding a bioluminescent polypeptide, and can be grown to produce plants that express the polypeptide. As such, the invention provides seeds produced by a transgenic plant obtained by a method of the invention.

Transgenic plants comprising different transgenes also can be crossbred, thereby providing a means to obtain transgenic plants containing two or more different transgenes, one of which encodes a bioluminescent polypeptide and the other or others of which confer a desirable characteristic to the plant. Methods for breeding plants and selecting for crossbred plants having desirable characteristics or other characteristics of interest are well known in the art.

A method of the invention can be performed by introducing a recombinant nucleic acid molecule of the invention into a plant cell. As used herein, the term “introducing” means transferring a polynucleotide into a plant cell. A nucleic acid molecule can be introduced into a cell by a variety of methods. For example, the recombinant nucleic acid molecule of the invention, which can be contained in a vector, can be introduced into a plant cell using a direct gene transfer method such as electroporation or microprojectile mediated transformation, or using Agrobacterium mediated transformation. As used herein, the term “transformed” refers to a plant cell containing an exogenously introduced nucleic acid molecule.

It should be recognized that one or more nucleic acid molecules, which are the same or different and at least one of which is a recombinant nucleic acid molecule of the invention, can be introduced into a plant cell, thereby providing a means to obtain a genetically modified plant cell containing multiple copies of a single transgene, or containing two or more different genes, either or both of which can be present in multiple copies. Such genetically modified plant cells can be produced, for example, by simply selecting plant cells having multiple copies of a single type of transgene; by cotransfecting plant cells with two or more populations of different transgenes and identifying those containing the two or more different transgenes; or by crossbreeding transgenic plants, each of which contains one or more desired transgenes, and identifying those progeny having the desired transgenes.

Methods for introducing a nucleic acid molecule into a plant cell to obtain a transformed plant also include direct gene transfer (see European Patent A 164 575), injection, electroporation, biolistic methods such as particle bombardment, pollen-mediated transformation, plant RNA virus-mediated transformation, liposome-mediated transformation, transformation using wounded or enzyme-degraded immature embryos, or wounded or enzyme-degraded embryogenic callus, and the like. Transformation methods using Agrobacterium tumefaciens tumor inducing (Ti) plasmids or A. rhizogenes root-inducing (Ri) plasmids, or other plant virus vectors are well known in the art (see, for example, WO 99/47552; Weissbach and Weissbach, “Methods for Plant Molecular Biology” (Academic Press, NY 1988), section VIII, pages 421-463; Grierson and Corey, “Plant Molecular Biology” 2d Ed. (Blackie, London 1988), Chapters 7-9, each of which is incorporated herein by reference; Horsch et al., supra, 1985). The wild type form of A. tumefaciens, for example, contains a Ti plasmid, which directs production of tumorigenic crown gall growth on host plants. Transfer of the tumor inducing T-DNA region of the Ti plasmid to a plant genome requires the Ti plasmid-encoded virulence genes as well as T-DNA borders, which are a set of direct DNA repeats that delineate the region to be transferred. An Agrobacterium based vector is a modified form of a Ti plasmid, in which the tumor inducing functions are replaced by a nucleotide sequence of interest that is to be introduced into the plant host.

Methods of using Agrobacterium mediated transformation include cocultivation of Agrobacterium with cultured isolated protoplasts; transformation of plant cells or tissues with Agrobacterium; and transformation of seeds, apices or meristems with Agrobacterium. In addition, in planta transformation by Agrobacterium can be performed using vacuum infiltration of a suspension of Agrobacterium cells (Bechtold et al., C.R. Acad. Sci. Paris 316:1194, 1993, which is incorporated herein by reference).

Agrobacterium mediated transformation can employ cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. Binary vectors are well known in the art (see, for example, De Framond, BioTechnology 1:262, 1983; Hoekema et al., Nature 303:179, 1983, each of which is incorporated herein by reference) and are commercially available (Clontech; Palo Alto Calif.). For transformation, Agrobacterium can be cocultured, for example, with plant cells or wounded tissue such as leaf tissue, root explants, hypocotyls, cotyledons, stem pieces or tubers (see, for example, Glick and Thompson, “Methods in Plant Molecular Biology and Biotechnology” (Boca Raton Fla., CRC Press-1993), which is incorporated herein by reference). Wounded cells within the plant tissue that have been infected by Agrobacterium can develop organs de novo when cultured under the appropriate conditions; the resulting transgenic shoots eventually give rise to transgenic plants, which contain an exogenously introduced circadian-regulated polynucleotide, or an oligonucleotide portion thereof.

Agrobacterium mediated transformation has been used to produce a variety of tratsgenic plants, including, for example, transgenic cruciferous plants such as Arabidopsis, mustard, rapeseed and flax; transgenic leguminous plants such as alfalfa, pea, soybean, trefoil and white clover; and transgenic solanaceous plants such as eggplant, petunia, potato, tobacco and tomato (see, for example, Wang et al., “Transformation of Plants and Soil Microorganisms” (Cambridge, University Press 1995), which is incorporated herein by reference). In addition, Agrobacterium mediated transformation can be used to introduce an exogenous nucleic acid molecule into apple, aspen, belladonna, black currant, carrot, celery, cotton, cucumber, grape, horseradish, lettuce, morning glory, muskmelon, neem, poplar, strawberry, sugar beet, sunflower, walnut, asparagus, rice and other plants (see, for example, Glick and Thompson, supra, 1993; Hiei et al., Plant J. 6:271-282, 1994; Shimamoto, Science 270:1772-1773, 1995).

Suitable strains of A. tumefaciens and vectors as well as transformation of Agrobacteria and appropriate growth and selection media are well known in the art (GV3101, pMK90RK), Koncz, Mol. Gen. Genet. 204:383-396, 1986; (C58C1, pGV3850kan), Deblaere, Nucl. Acid Res. 13:4777, 1985; Bevan, Nucleic Acid Res. 12:8711, 1984; Koncz, Proc. Natl. Acad. Sci. USA 86:8467-8471, 1986; Koncz, Plant Mol. Biol. 20:963-976, 1992; Koncz, Specialized vectors for gene tagging and expression studies. In: Plant Molecular Biology Manual Vol. 2, Gelvin and Schilperoort (Eds.), Dordrecht, The Netherlands: Kluwer Academic Publ. (1994), 1-22; European Patent A-1 20 516; Hoekema: The Binary Plant Vector System, Offsetdruikkerij Kanters B. V., Alblasserdam (1985), Chapter V; Fraley, Crit. Rev. Plant. Sci., 4:1-46; An, EMBO J. 4:277-287, 1985).

Where the recombinant nucleic acid molecule is contained in a vector, the vector can contain functional elements, for example “left border” and “right border” sequences of the T-DNA of Agrobacterium, which allow for stable integration into a plant genome. Furthermore, methods and vectors that permit the generation of marker-free transgenic plants, for example, where a selectable marker gene is lost at a certain stage of plant development or plant breeding, are known, and include, for example, methods of co-transformation (Lyznik, Plant Mol. Biol. 13:151-161, 1989; Peng, Plant Mol. Biol. 27:91-104, 1995), or methods that utilize enzymes capable of promoting homologous recombination in plants (see, e.g., W097/08331; Bayley, Plant Mol. Biol. 18:353-361, 1992; Lloyd, Mol. Gen. Genet. 242:653-657, 1994; Maeser, Mol. Gen. Genet. 230:170-176, 1991; Onouchi, Nucl. Acids Res. 19:6373-6378, 1991; see, also, Sambrook et al., supra, 1989).

A direct gene transfer method such as electroporation also can be used to introduce a polynucleotide into a cell such as a plant cell. For example, plant protoplasts can be electroporated in the presence of a recombinant nucleic acid molecule comprising a nucleotide sequence encoding a bioluminescent polypeptide, which can be in a vector (Fromm et al., Proc. Natl. Acad. Sci., USA 82:5824, 1985, which is incorporated herein by reference). Electrical impulses of high field strength reversibly permeabilize membranes allowing the introduction of the nucleic acid. Electroporated plant protoplasts reform the cell wall, divide and form a plant callus. Microinjection can be performed as described in Potrykus and Spangenberg (eds.), Gene Transfer To Plants. Springer Verlag, Berlin, N.Y. (1995). A transformed plant cell containing the introduced recombinant nucleic acid molecule can be identified due to the presence of a selectable marker included in the construct, or simply by looking at the plant cells and observing visible bioluminescence.

Microprojectile mediated transformation also can be used to introduce a recombinant nucleic acid molecule of the invention into a plant cell (Klein et al., Nature 327:70-73, 1987, which is incorporated herein by reference). This method utilizes microprojectiles such as gold or tungsten, which are coated with the desired nucleic acid molecule by precipitation with calcium chloride, spermidine or polyethylene glycol. The microprojectile particles are accelerated at high speed into a plant tissue using a device such as the BIOLISTIC PD-1000 particle gun (BioRad; Hercules Calif.).

Microprojectile mediated delivery (“particle bombardment”) is especially useful to transform plant cells that are difficult to transform or regenerate using other methods. Methods for the transformation using biolistic methods are well known (Wan, Plant Physiol. 104:37-48, 1984; Vasil, BioTechnology 11:1553-1558, 1993; Christou, Trends in Plant Science 1:423-431, 1996). Microprojectile mediated transformation has been used, for example, to generate a variety of transgenic plant species, including cotton, tobacco, corn, hybrid poplar and papaya (see Glick and Thompson, supra, 1993). Important cereal crops such as wheat, oat, barley, sorghum and rice also have been transformed using microprojectile mediated delivery (Duan et al., Nature Biotech. 14:494-498, 1996; Shimamoto, Curr. Opin. Biotech. 5:158-162, 1994). A rapid transformation regeneration system for the production of transgenic plants such as a system that produces transgenic wheat in two to three months (see European Patent No. EP 0709462A2, which is incorporated herein by reference) also can be useful for producing a transgenic plant according to a method of the invention, thus allowing more rapid identification of gene functions. The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, Agrobacterium mediated transformation, and the like.

Plastid transformation also can be used to introduce a nucleic acid molecule into a plant cell (U.S. Pat. Nos. 5,451,513, 5,545,817, and 5,545,818; WO 95/16783; McBride et al., Proc. Natl. Acad. Sci., USA 91:7301-7305, 1994). Chloroplast transformation involves introducing regions of cloned plastid DNA flanking a desired nucleotide sequence, for example, a selectable marker together with polynucleotide of interest into a suitable target tissue, using, for example, a biolistic or protoplast transformation method (e.g., calcium chloride or PEG mediated transformation). One to 1.5 kb flanking regions (“targeting sequences”) facilitate homologous recombination with the plastid genome, and allow the replacement or modification of specific regions of the plastome. Using this method, point mutations in the chloroplast 16S rRNA and rps12 genes, which confer resistance to spectinomycin and streptomycin and can be utilized as selectable markers for transformation (Svab et al., Proc. Natl. Acad. Sci., USA 87:8526-8530, 1990; Staub and Maliga, Plant Cell 4:39-45, 1992), resulted in stable homopiasmic transformants, at a frequency of approximately one per 100 bombardments of target leaves. The presence of cloning sites between these markers allowed creation of a plastid targeting vector for introduction of foreign genes (Staub and Maliga, EMBO J. 12:601-606, 1993). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab and Maliga, Proc. Natl. Acad. Sci., USA 90:913-917, 1993). Approximately 15 to 20 cell division cycles following transformation are generally required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein.

Plants suitable for treatment according to a method of the invention so as to be visibly bioluminescent can be monocots or dicots and include, but are not limited to, maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, Arabidopsis thaliana, and woody plants such as coniferous and deciduous trees. Thus, a transgenic plant or genetically modified plant cell of the invention can be an angiosperm or gymnosperm.

Angiosperms are divided into two broad classes based on the number of cotyledons, which are seed leaves that generally store or absorb food; a monocotyledonous angiosperm has a single cotyledon, and a dicotyledonous angiosperm has two cotyledons. Angiosperms produce a variety of useful products including materials such as lumber, rubber, and paper; fibers such as cotton and linen; herbs and medicines such as quinine and vinblastine; ornamental flowers such as roses and, where included within the scope of the present invention, orchids; and foodstuffs such as grains, oils, fruits and vegetables.

Angiosperms encompass a variety of flowering plants, including, for example, cereal plants, leguminous plants, oilseed plants, hardwood trees, fruit-bearing plants and ornamental flowers, which general classes are not necessarily exclusive. Cereal plants, which produce an edible grain cereal, include, for example, corn, rice, wheat, barley, oat, rye, orchardgrass, guinea grass, sorghum and turfgrass. Leguminous plants include members of the pea family (Fabaceae) and produce a characteristic fruit known as a legume. Examples of leguminous plants include, for example, soybean, pea, chickpea, moth bean, broad bean, kidney bean, lima bean, lentil, cowpea, dry bean, and peanut, as well as alfalfa, birdsfoot trefoil, clover and sainfoin. Oilseed plants, which have seeds that are useful as a source of oil, include soybean, sunflower, rapeseed (canola) and cottonseed.

Angiosperms also include hardwood trees, which are perennial woody plants that generally have a single stem (trunk). Examples of such trees include alder, ash, aspen, basswood (linden), beech, birch, cherry, cottonwood, eln, eucalyptus, hickory, locust, maple, oak, persimmon, poplar, sycamore, walnut, sequoia, and willow. Trees are useful, for example, as a source of pulp, paper, structural material and fuel.

Angiosperms are fruit-bearing plants that produce a mature, ripened ovary, which generally contains seeds. A fruit can be suitable for human or animal consumption or for collection of seeds to propagate the species. For example, hops are a member of the mulberry family that are prized for their flavoring in malt liquor. Fruit-bearing angiosperms also include grape, orange, lemon, grapefruit, avocado, date, peach, cherry, olive, plum, coconut, apple and pear trees and blackberry, blueberry, raspberry, strawberry, pineapple, tomato, cucumber and eggplant plants. An ornamental flower is an angiosperm cultivated for its decorative flower. Examples of commercially important ornamental flowers include rose, lily, tulip and chrysanthemum, snapdragon, camellia, carnation and petunia plants, and can include orchids.

The skilled artisan will recognize that the methods of the invention can be practiced using these or other angiosperms, as desired, as well as gymnosperms, which do not produce seeds in a fruit. As such, the methods of the invention can be used to prepare bioluminescent plants of various families, including, for example, Asteraceae-Aster family; Fabaceae-Pea family; Rubiaceae-Madder family; Poaceae-Grass family; Euphorbiaceae-Spurge family; Lamiaceae-Mint family, Melastomataceae-Melastome family; Liliaceae-Lily family; Scrophulariaceae-Figwort family; Acanthaceae-Ancanthus family, Myaceae-Myrtle family, Cyperaceae-Sedge family; Ericaceae-Heath family, Apiaceae-Carrot family, Rosaceae-Rose family; Brassicaceae-Mustard family, Araceae-Arum family, Asclepiadaceae-Milkweed family; Arecaceae-Pahm family, Solanaceae-Potato family, Boraginaceae-Borage family; Gesneriaceae-Generiad family, Lauraceae-Laurel family, and Bromeliaceae-Bromeliad family. In certain embodiments, the methods of the invention also can be used to prepare bioluminescent plants of Orchidaceae-Orchid family, whereas in other embodiments, members of the orchid family are specifically excluded.

A recombinant nucleic acid molecule of the invention or a vector containing the recombinant nucleic acid molecule provides a valuable reagent for identifying plant cells containing an introduced nucleic acid molecule. For example, the recombinant nucleic acid molecule, or vector containing the recombinant nucleic acid molecule, can include a nucleotide sequence of interest. Following transformation of plant cells with the recombinant nucleic acid molecule of the invention, which contains the nucleotide sequence of interest, visibly bioluminescent plant cells can be selected simply by looking at the plant cells, for example, under a microscope, and picking out the ones that are visibly bioluminescent, thereby selecting plant cells that also contain the nucleotide sequence of interest. Thus, a recombinant nucleic acid molecule of the invention is useful as a selectable marker, and provides a quick, efficient, and inexpensive means to identify a plant cell containing a nucleotide sequence of interest.

A nucleotide sequence of interest also can encode an immunogenic polypeptide, for example, a viral or bacterial polypeptide, or a peptide portion thereof. A genetically modified plant cell, particularly a transgenic plant comprising such a plant cell, can be used as a source of a vaccine, wherein the individuals to be immunized merely ingest the plant, and a protective or palliative immune response is obtained. As an additional advantage, problems generally associated with the preparation and purification of a vaccine, as well as storage and refrigeration problems and the like are obviated. In view of these examples, it will be recognized that genetically modified plant cells and transgenic plant comprising such cells containing essentially any nucleotide sequence of interest can be prepared, and that the inclusion of a recombinant nucleic acid molecule of the invention provides a selectable marker that facilitates the identification of a transformed plant cell or plant comprising such a cell that contains the nucleotide sequence of interest

A recombinant nucleic acid molecule of the invention, or a vector containing such a nucleic acid molecule, also is useful for preparing transgenic plants having great ornamental value. Visibly bioluminescent plants containing genetically modified plant cells have not previously been described. As such, the transgenic plants of the invention, which “glow in the dark,” provide a novel ornamental plant. For example, a transgenic plant expressing a luciferase polypeptide or variant thereof can be sprayed with water containing luciferin, or the transgenic plant or a cutting or other portion of the transgenic plant, for example, an inflorescence of the plant, can be dipped or placed in a solution containing luciferin, whereupon the visible bioluminescence is produced. For example, flowers can be cut from a transgenic plant of the invention and placed in water containing luciferin, wherein the luciferin is taken up and transported to the flower, thus providing a visibly bioluminescent flower. Generally, the amount of luciferin sufficient to induce visible bioluminescence includes at least about 0.1 mM, and concentrations from about 0.1 mM to 5 mM or more can be used. In addition, the solution containing luciferin can further include a surfactant, for example, a non-ionic detergent such as TRITON X-100 detergent or NONIDET P40 detergent in a concentration of about 0.001% to about 0.1%, thus facilitating entry of the luciferin into the plant cells. Significantly, luciferase-mediated bioluminescence continues for several hours following a single administration of luciferin and, therefore, there is no requirement that the plants be continually treated to maintain bioluminescence. Furthermore, the transgenic plants can be selected based on their expression of variable levels of light, including plants that glow dimly, yet visibly, and those that glow very brightly.

In addition to providing general ornamental value, the visibly bioluminescent transgenic plants of the invention also can provide a practical value. For example, visibly bioluminescent transgenic plants that grow as bushes, shrubs or trees can be arranged along a walkway or driveway. Where such plants express luciferase or a variant thereof, for example, the plants can be watered in the early evening with a solution containing luciferin such that they illuminate the walkway or driveway after night falls. Thus, the transgenic plants of the invention provide the additional advantage they can be used to make an area safer by illuminating the area.

The present invention also relates to kit, which contains a genetically modified plant cell of the invention, or a derivative of the genetically modified plant cell, for example, a transgenic plant derived from the genetically modified plant cell, or a cell, a tissue, or an organ of such a transgenic plant. As such, a kit of the invention can contain one or more flowers, bracts, leaves or other tissues or organs, which, upon contact with luciferin, are visibly luminescent.

Accordingly, a kit of the invention can further include an amount of luciferin sufficient for generating visible bioluminescence of the genetically modified plant cell or the derivative of the genetically modified plant cell, and also can include a plurality of such amounts of luciferin, thus allowing the generation of visible bioluminesce a number of times, as desired. In addition, a kit of the invention can include an amount of a carotenoid inhibitor, for example, norfluorazon, sufficient for reducing or inhibiting chlorophyll production in the genetically modified plant cell or the derivative of the genetically modified plant cell.

In another embodiment, a kit of the invention contains one or more cuttings, seeds, or other portion or derivative of a visibly bioluminescent plant of the invention such that a visibly luminescent transgenic plant can be grown therefrom. As such, the kit also can include reagents for growing a transgenic plant from the cutting or seed, including, for example, a suitable plant food or other nutrient source required for growth of the particular plant. In addition, the kit can include an amount of a carotenoid inhibitor sufficient for reducing or inhibiting chlorophyll production in a transgenic plant grown from the cutting or seed; and/or can include an amount of luciferin sufficient for generating visible bioluminescence of a transgenic plant grown from the cutting or seed.

Generally, a kit of the invention includes a container for the components of the kit. It should be recognized, however, that the container can be any convenient means for holding a component of the kit, including, for example, a packet or collection of packets containing seeds obtained from a transgenic plant of the invention, or containing predetermined amounts of a reagent such as luciferin, which can be added to a specified amount of water to provide a solution sufficient to generate visible bioluminescence from a composition of the invention. In one embodiment, the kit comprises a flower cutting of a transgenic plant of the invention in a vase, and can further include one or more predetermined amounts of a reagent such as luciferin. In another embodiment, the kit comprises a seed or a cutting obtained from a transgenic plant of the invention, and further includes a reagent such as luciferin and/or norfluorazon, in which the seed or cutting, or a plant derived therefrom, can be grown.

The following examples are intended to illustrate but not limit the invention.

### EXAMPLE 1

### Preparation of Luciferase Expression Vectors

This example provides a method for preparing luciferase constructs that can be incorporated into an expression vector and are useful for producing a visibly bioluminescent plant.

Plasmid pRTL2, which contains a CaMV 35S promoter with duplicated enhancer, a TEV leader and initial coding sequence from pTL-7SN, and a CaMV 35S polyadenylation (poly A) signal sequence (Topfer et al., Nucl. Acids Res. 15:5890, 1987; Restrepo et al., Plant Cell 2:987, 1990, each of which is incorporated herein by reference; see, also, Carrington and Freed, supra, 1990) was sequentially digested with Nco I and Xba I, then separated by electrophoresis on a 0.8% agarose gel. The nucleotide sequence of the CaMV 35S promoter with duplicated enhancer, TEV leader and initial coding sequence, and CaMV 35S poly A signal sequence of pRTL2 is shown as SEQ ID NO:3. A DNA band of approximately 3.9 kb representing the linear pRTL2 (see nucleotides 1 to 1188 and 2844 to 5475 of SEQ ID NO:1) was isolated and extracted from the gel using a QIAQUICK DNA gel extraction kit (Qiagen; Valencia Calif.) according to manufacturer's instructions. The Nco I site in the TEV leader contains the start codon for translation. Translational fusions containing the initial 20 amino acid residues of the TEV polyprotein are made by ligation to one of the polylinker sites, provided care is taken to avoid the STOP codon in the Xba I site (see SEQ ID NO:3).

In a parallel reaction, pGL3 (Promega Corp., Madison Wis.), which contains a modified luciferase coding sequence, luc⁺ (Groskreutz et al., Promega Notes 50:2, 1995; U.S. Pat. No.5,670,356, each of which is incorporated herein by reference) was digested sequentially with Xba I and Nco I, then separated by electrophoresis on a 0.8% agarose gel. Two bands were present—a 1.6 kb band representing the luc⁺ coding sequence (see U.S. Pat. No.5,670,356) and an approximately 3 kb band representing the remainder of the pGL3 plasmid. The approximately 1.6 kb luc⁺ band (see nucleotides 1188 to 2844of SEQ ID NO:1) was isolated and extracted as described above.

The isolated luc⁺ sequence (4 μl) was ligated into the linearized pRTL2 vector (1 μl) using ligase and ligation buffer from BRL (Gaithersburg Md.). The ligation was performed overnight at 16° C. E. coli DH5α cells were transformed using a BioRad Electroporator in a ratio of 2 μl of ligated DNA to 20 μl of cells. Following electroporation, the transformed cells were incubated in LB medium for 1 hr at 37° C., then were plated on LB plates containing 50 μg/ml ampicillin. Of approximately 3000 colonies, 32 were selected and screened by PCR for the luc⁺ insert. PCR primers were specific for the 5′ end of the TEV promoter in pRTL2 and for the 3′ end of the poly A terminator in pRTL2 (see SEQ ID NO:3). PCR analysis revealed that 30 of the colonies contained the luc⁺ insert. The presence of the insert in 6 clones was verified by restriction analysis, and two candidate clones, designated TK1-7 and TK1-8, were sequenced to confirm the presence of the luc⁺ insert.

The TK1-8 clone was digested at 37° C. for 2 hr with Hind III, and the fragment containing the CaMV 35S promoter with duplicated enhancer, TEV leader, luc⁺ coding sequence and poly A termination signal (designated “TK1829”; see FIG. 2, and nucleotides 8366 to 11,113 of SEQ ID NO:2) was isolated by electrophoresis as described above. The binary vector, pPZP221, also was digested with Hind III and isolated. The linearized pPZP221 vector was treated with shrimp alkaline phosphatase for 1 hr at 37° C., to remove terminal phosphate groups, followed by 15 min at 65° C. to heat inactivate the enzyme, then the TK1829 insert and linearized pPZP221 vector were ligated and transformed into DH5α cells as described above. Cells were plated on LB agar plates containing X-gal, IPTG, and 100 μg/ml spectinomycin.

Forty-six white colonies were selected, and the presence of the luc⁺ coding sequence was confirmed by PCR as described above; all were positive for the luc⁺ sequence. Six samples were selected and the orientation of the insert was determined by restriction digest analysis, using Sca I or Nar I. One construct (#29) having the correct orientation was selected. The binary vector containing the luc⁺ insert was designated pPZPTK1829 (FIG. 2; SEQ ID NO:2), and the inserted expression construct was designated TK1829 (nucleotides 8366 to 11,113 of SEQ ID NO:2). TK1829 was sequenced using primers specific for 5′ and 3′ insert flanking regions of the pPZP221 vector. The pRTL2 vector from which the TK1829 insert was obtained was designated pTK1829 (see FIG. 1; see, also, SEQ ID NO:1).

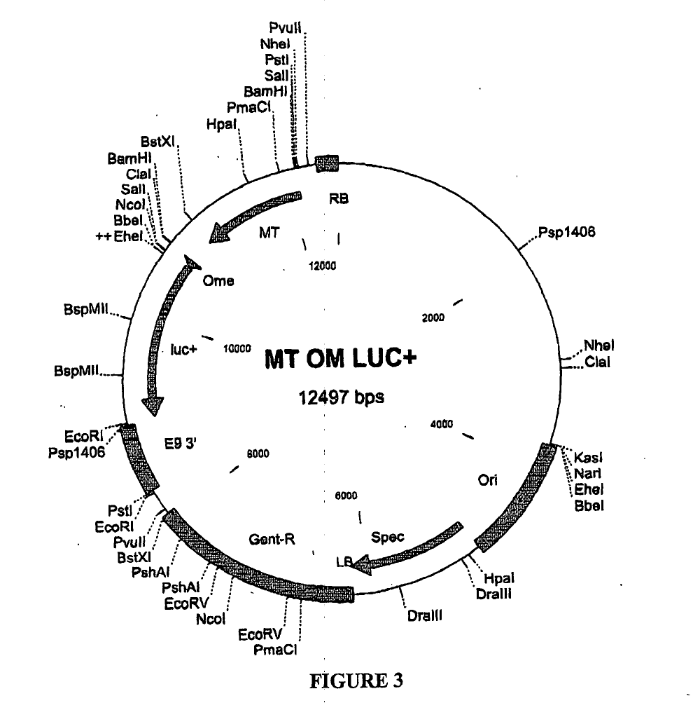

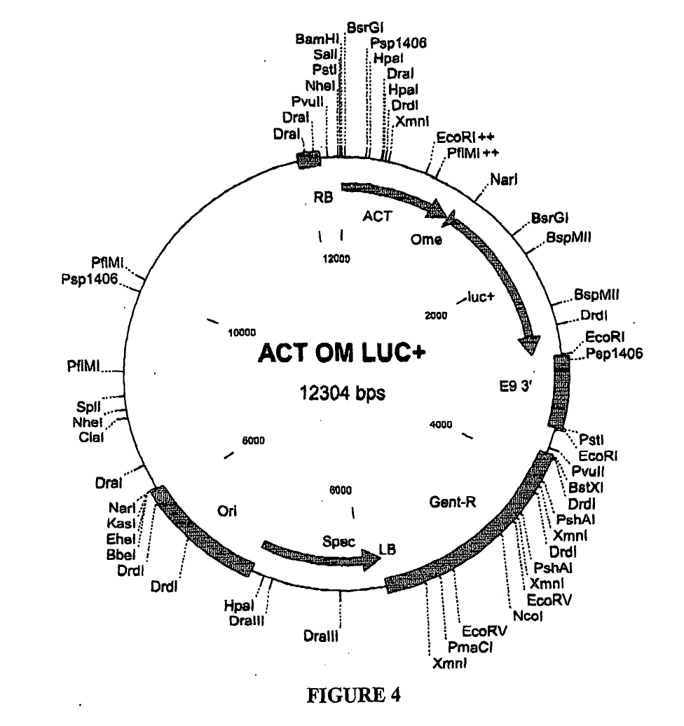

The pMT-OM-LUC⁺ (SEQ ID NO:4; FIG. 3) and pACT-OM-LUC⁺ (SEQ ID NO:5; FIG. 4) binary vector plasmids were prepared from the pPZP221 binary vector, as described above. The metallothionein (MT) or actin (ACT) promoter was inserted immediately preceding the omega enhancer in a Bam HI site, which provides a convenient site to substitute other transcriptional regulatory elements.

### EXAMPLE 2

### Preparation and Characterization of Visibly Bioluminescent Plants

This example provides a method for introducing a luciferase expression construct into plants such that a genetically modified visibly bioluminescent plant is produced, and a method for determining a threshold value of photon emission required for visible luminescence.

A. Preparation of TK1829 Transformed Plants

The TK1829 expression construct was introduced from pPZPTK1829 into Agrobacterium l strain ABI 1 with the help of pRK2013 by tri-parental mating. Agrobacterium containing the TK1829 construct were selected based on their resistance to the 100 μg/ml spectinomycin, 50 μg/ml kanamycin, and 25 μg/ml chloramphenicol. TK1829-Agrobacterium strains were grown, then DNA was purified and checked by restriction digest analysis using Sca I (2 hr; 37° C.) to verify the presence of the insert.

Briefly, TK1829-Agrobacterium was grown as described below, and 28 day old plants were infiltrated. Supernova (Snv) strain plants and Columbia (wild type) plants were each infiltrated. Two sets of infiltrations were performed on Columbia and Snv plants. Each time, two 4 inch pots of each line were used and each pot contained approximately 50 plants, for a total of 200 plants per line infiltrated.

For in planta plant transformation, 4 inch pots were prepared with Scott's Metro mix 200 soil and covered with window screen mesh. Soil was wet and mounded up, and the screen mesh was secured by wrapping a rubber band around the neck of the pot, maling sure that the mesh was in contact with the soil surface so that germinating seedlings could get through the mesh. Seeds were stratified in 10 to 50 ml of sterile water and stored at 4° C. for 3 days prior to being applied to pots. Seeds were sown using a dropper, to spread seeds evenly over the surface of the mesh. Plants were grown at approximately 22° C., in 12 hr light:12 hr dark per day to a stage at which bolts were anywhere from 3 to 10 inches high. The bolts were cut off to induce growth of secondary inflorescence.

Infiltration was most effective 4 to 5 days after decapitation of bolts, but can be performed up to 8 days later. For infiltration, 5 to 20 ml liquid preculture of Agrobactenium containing the appropriate construct were grown for about 2 days in a shaking incubator at 28° C., then 400 ml of LB was inoculated with the preculture and incubate for one additional day. The large culture generally was prepared the day before the infiltration was performed.

Transformed Agrobacterium cells were grown to an optical density at 600 nm (OD600) of approximately 1.2, then harvested by centrifugation (4000 rpm, 10 min, room temperature), and resuspended in infiltration medium (1/2×Murashige and Skoog salts; 1×Gamborg's vitamins; 5.0% sucrose; 0.44 μM benzylamino-purine (10 μg per liter of a 1 ng/ml stock); pH adjusted to 5.7 using KOH; then add 200 μl/l Silwet; media need not be sterilized if used immediately) to an OD 600 of approximately 0.8. The OD600 of an overnight culture generally was about 1.2 to 1.6 units. A 400 ml suspension generally was adequate for an infiltration procedure. The same suspension can be used as many as three times.

About 150-200 ml of the Agrobacterium suspension was added to either a beaker or dish and the prepared plants were inverted into the suspension. The plants were entirely submerged and kept in the solution for 10 to 15 min. The pots then were removed from the suspension and drained thoroughly, laid on their side in a plastic flat, and covered with a plastic dome or Saran Wrap to maintain humidity. The flat was uncovered the next day and the pots set upright. A little water was added to the bottom of the tray to help keep the humidity level high while the plants were recovering. The plants were grown for 3 to 4 weeks, keeping the bolts from each pot together and separated from neighboring pots and different infiltrations. Once plants began to yellow and senesce, watering was reduced or discontinued. Seeds were harvested together from one pot.

Transformants were selected on 150×15mm selection plates (1/2×Murashige and Skoog salts; 1×Gamborg's vitamins; 1% sucrose; 0.5 g/l MES; 0.8% agarose; pH to 5.7; autoclaved, then add 75 μg/ml gentamycin, 500 μg/ml carbenicilin, 50 μg/ml kanamycin). Seeds were surface sterilize for 2 min in 500 μl 70% ethanol; followed by 10 min in 40% bleach, 1% SDS, 59% water, then rinsed 3 times with sterile water. The seeds were resuspended in 3 to 4 ml of sterile water and plated by pouring onto a plate and shaking until evenly distributed. The plates were allowed to sit for 5 to 10 min, then excess liquid was removed. Approximately 70 to 100 μl of dry seeds were added per plate. Plants were allowed to stratify at 4° C. for 3 days, then transferred to a growth chamber.

In some experiments, the herbicide, norflurazon (5 μM; 4-chloro-5-(methylamino)-2-{3-(tri-fluoromethyl)phenyl}-3(2H)-pyridazinone) was included in the growth medium. Norflurazon inhibits chlorophyll synthesis, resulting in the growth of “white” plants. Treatment of plants with norflurazon resulted in greater emission of visible bioluminescence due to the lack of chlorophyll, which absorbs light and blocks light emission (see Tables).

After 7 to 10 days (depending on the antibiotic resistance selected for), transformants were identified as dark green plants with elongated roots that penetrated the agar. These seedlings were transferred onto a second set of selection plates. After several days, plants had 4 to 8 true leaves and branching roots. Pseudo-resistant plants were unable to survive in this medium. The plants then were transferred to soil, and kept covered for the first few days.

Plants were allowed to recover, bolt, set seed and senesce. Seeds were harvested when dry, then were further dried in a dessicator for 1 week. Yield was approximately 0.5 to 0.75 ml of seed per pot. T1 (transgenic first generation) seeds in the Columbia background were screened on 75 μg/ml gentamycin and 500 μg/ml carbenicilin MS plates. T1 seeds in the Snv background were screened on 75 μg/ml gentamycin, 500 μg/ml carbenicilin, and 50 μg/ml kanamycin (Snv strain contains a transgene conferring kanamycin resistance).

Over an 8 week period, 1.6 ml (approximately 32,000 seeds) of each T1 seed set was sterilized and planted in 200 μl (4000 seeds) aliquots on 50 ml plates as described above. Plants were stratified for 3 days at 4° C., then moved to a growth chamber set for 12 hr light: 12 hr dark and approximately 27° C. Sixty-seven T1 seedlings in the Snv background and 25 T1 seedlings in the Columbia background grew under the selection conditions, and were recovered and transferred to soil to harvest T2 seed. It took approximately 12 to 16 weeks to take T1 seed to T2 seed, and varied between plants.

B. Characterization of TK1829 Transformed Plants

T2 seeds of Columbia and Supernova were sterilized and plated as above, and screened for antibiotic resistance. In addition, the seedlings were screened for bioluminescence, visually and with the Hamamatsu photon counting camera. T2 seedlings within individual families were selected based on their intensity and brightness. Three lines in the Snv background (Snv 11, Snv 37 and Snv 57) and two lines in the Columbia background (Col 10 and Col 19) were selected as the most visibly bioluminescent plants. Seventy-eight T3 individuals of Snv 11, which was extremely bright, were selected based on their visible bioluminescence. Three of these (Snv 11-8, Snv 11-48, Snv 11-52) were selected as the brightest and were maintained as individual lines.

For characterization of luciferase expression, tissue was collected from 22 day old plants grown under the same conditions. Examination of Columbia wild-type plants, Columbia plants transformed with 35S:Luc (“35S:Luc”) and Snv plants transformed with cab2:Luc (“Supernova”; see Hicks et al., supra, 1996) was included for comparison. In various assays (see below), protein concentration was determined, relative light units (RLU) were acquired using a luminometer, western blot analysis was performed using recombinant luciferase antibodies, plants were imaged using a Night Owl Camera (see below) to obtain counts of light/mm²/sec, and plants were inspected visually to confirm that bioluminescence was visible to the eye. Based on these assays, the approximate amounts of luciferase per microgram of total protein and the fold increase in enzyme activity were determined, and the amount of light emitted by the plants was determined.

For protein assays, a few hundred 22 day old seedlings from plants grown under the same conditions were placed in liquid nitrogen, ground into a fine power, and stored in aliquots of approximately 200 μl volume on dry ice or at −80° C. until the time proteins were extracted. Approximately 100 mg of the powdered tissue was extracted in 500 μl of 1×Promega Cell Lysis Reagent (CLR), ground briefly with a mortar and pestle, then centrifuged at 4° C. for 5 min 14,000 rpm. The supernatant was divided into 100 μl aliquots and transferred immediately to dry ice. Any samples not used within 1 hr were stored at −80° C. to preserve enzyme activity. Protein concentrations were determined for 20 μl of tissue extract using the DC Protein Assay Reagent (BioRad); optical density (OD) was determined at 750 nm using a Pharmacia Ultrospec III spectrophotometer.

Acquired Relative Light Units (RLU) were obtained using a Turner TD20E Luminometer. Samples from plants containing the TK1829 construct were diluted 1/100 (5 μl/500 μl) in order for the reading to be within the range of the luminometer. Five μl of sample and 50 μl of Promega Luciferase Assay Reagent (LAR) was used. The maximum value of the range of readings taken every 30 sec for the first 5 min of the reaction was entered into the data tables. The amount of luciferase was approximated by western blot analysis (Tables 1 and 2) or using the RLU data obtained by luminometry and the total protein concentration obtained by spectrophotometry (Table 4).

Western blot analysis of the extracts was performed using enhanced chemifluoresence (ECF; Amersham) to visualize protein bands. For samples containing the TK1829 construct, 0.1 μg of total protein was used. For all other samples, 1.0 μg of total protein was used. The 10-fold difference was necessary to keep the luciferase protein concentrations within the range of the assay. A luciferase primary antibody and an alkaline phosphatase secondary antibody (Promega) were used to identify protein bands. Proteins were separated by electrophoresis on, 10% SDS PAGE gels (BioRad), then transferred to Hybond nitrocellulose. Images were visualized using a Molecular Dynamics Fluorimeter, and data was analyzed using ImageQuant software.

Luciferase was expressed in 35S:Luc transformed Columbia plants (“35S:Luc”) and cab2:Luc transformed Supernova plants (“Supernova”; Table 1), but these plants were not visibly bioluminescent when examined by eye. In comparison, the TK1829 transformed Columbia plants were visibly bioluminescent. These results indicate that the threshold for visible bioluminescence is between about 85 and 125 pg luc⁺ protein/μg protein as determined by western blot analysis (Table 1).

The TK1829 transformed Supernova plants expressed about 6.5-fold more luc⁺ than the TK1829 transformed Columbia plants as determined by western blot analysis (Table 1). In addition, the Snv-TK1829 plants emitted about 7-fold more photons of light/mm²/sec than the Col-TK1829 plants. These results demonstrate that visible bioluminescence can be produced in various Arabidopsis strains. In addition, growth of the TK1829 transformed Supernova plants in norflurazon resulted in an almost 2-fold increase in luciferase protein as measured by luminometry, and a greater than 7-fold amount of light emitted as measured using the Night Owl imaging system.

This result demonstrates that the amount of visible bioluminescence can be increased by inhibiting chlorophyll synthesis in the transgenic plants.

TABLE 1

| | | pg Luciferase |
|---|---|---|
| Plant Line | | protein/μg total protein |
| a) | Columbia Wild Type | 0.01 |
| b) | 35S:Luc transformed Columbia | 7.0 |
| c) | cab2:Luc transformed Supernova | 85.0 |
| d) | TK1829 transformed Columbia | 125.0 |
| e) | TK1829 transformed Supernova | 834.0 |
| f) | TK1829 transformed Supernova with | 1456.0 |
| | Norflurazon | |

A comparison of western blot data on various plant extracts allowed a determination of the relative difference in luciferase protein concentration among the various plant lines (Table 2). Western blot analysis confirmed that Arabidopsis transformed with the TK1829 construct expressed a much greater amount of luciferase expression as compared to plants transformed with a 35S:Luc construct (see Table 2b and d) and as compared to cab2:Luc-transformed Snv (“Supernova”; see Table 2, c and e).

The various plant lines also were examined by imaging analysis using the EGG Berthold Night Owl imaging system (see, also, Example 2C, below). Plants were sprayed with approximately 1 ml of 5 mM luciferin in a solution of 0.001% TRITON X-100 detergent. The camera settings were kept consistent for all plant lines. However, the time duration for taking the images was varied to accommodate the different levels of bioluminescence in the various plant lines. All data was processed and averaged for photon counts of light/area over the time the image was taken (mm/sec); variation between individual plants was eliminated by analyzing pools of plants.

TABLE 2

| | | Fold increase in luciferase |
|---|---|---|
| Plant Comparison | | protein concentration |
| a) | Supernova vs. 35S:Luc | 5 |
| b) | TK1829 transformed Columbia | 128 |
| | vs. 35S:Luc | |
| c) | TK1829 transformed Columbia vs. | 31 |
| | Supernova | |
| d) | TK1829 transformed Supernova vs. | 244 |
| | 35S:Luc | |
| e) | TK1829 transformed Supernova vs. | 53 |
| | Supernova | |
| f) | TK1829 transformed Supernova with | 2 |
| | Norflurazon vs. the same line without | |
| g) | TK1829 transformed Supernova with | 450 |
| | Norflurazon vs. 35S:Luc | |

In parallel with the levels of luc⁺ expression as disclosed above, light emission by the TK1829 transformed plants was substantially greater than that of Columbia plants transformed with the 35S:Luc construct (“35S:Luc”) or of Snv plants transformed with a cab2:Luc construct (“Supernova”; see Table 3). Light emission of Arabidopsis plants transformed with TK1829, as measured by photon counts per area per second, was increased by as much as 5,200 fold over 35S:Luc transformed Columbia plants (see Table 3g).

Plants also were inspected by eye to confirm that they were visibly bioluminescent. The TK1829 transformed Columbia and TK1829-transformed Supernova plants were visibly bioluminescent (whether grown in the presence or absence of norflurazon), whereas the none of the other plants, including the 35S:Luc transformed Columbia and cab2:Luc transformed Supernova plants, were visibly bioluminescent. These results demonstrate that transformation of a plant with a construct that can express about 125 pg luc⁺/μg protein as determined by western blot analysis can effectively generate visibly bioluminescent plants.

TABLE 3

| | | Fold increase in light |
|---|---|---|
| Plant Comparison | | (cts/mm²/sec) |
| a) | Supernova vs. 35S:Luc | 70 |
| b) | Tk1829 transformed Columbia vs. 35S:Luc | 106 |
| c) | Tk1829 transformed Columbia vs. | 1.5 |
| | Supernova | |
| d) | Tk1829 transformed Supernova vs. | 712 |
| | 35S:Luc | |
| e) | TK1829 transformed Supernova vs. | 10 |
| | Supernova | |
| f) | TK1829 transformed Supernova with | 7 |
| | Norflurazon vs. Supernova | |
| g) | TK1829 transformed Supernova with | 5,200 |
| | Norflurazon vs. 35S:Luc | |

C. Threshold Photon Emission for Visible Bioluminescence

In order to provide a standard for determining the threshold number of visible light photons required for visible bioluminescence, the number of photons of visible light emitted per square millimeter per second was determined using a defined system. A Night Owl camera with WinLight Program LB981 (EG&G Berthold; Bad Wildbad, Germany) was used, and all images were acquired under the following conditions: Cosmic Suppression, ON; Defect Correction, ON; Star Killer, OFF; Camera Readout, SLOW; Gain, HIGH; Look up Table set to GAMMA. Measurements were taken in a photography dark room with a plate-to-lens distance of 275 mm (standard setting on Night Owl camera). Each plant was imaged for a time appropriate to the amount of bioluminescence emitted and values were normalized to photon counts/area/second.

Approximate photons/mm²/sec were calculated using a conversion of discharges per minute divided by counts per minute. The Night Owl camera system was calibrated using manufacturer-provided standards from the Beckman LS6500 scintillation system. Two standards were used. The first standard contained scintillant alone, which represents background levels of discharges per minute (dpm). The second standard contained an amount of carbon-14 in scintillant that produces 100,600 dpm. Images were taken using 1.1 pixel binning with background subtraction, and values of counts per second (cps) were acquired. The cps then was multiplied by 60 to obtain counts per minute (cpm). Each discharge is representative of the energy released by a disintegrating beta particle in the form of a photon. “Dpm” divided by “cpm” then provided an approximate efficiency for the camera, which was used for all data values to estimate actual photons emitted. For example, where the carbon-14 standard=3.6 cps and scintillant alone=2.2 cps, the difference=1.4 cps (actual cps)=84 cpm; and 100,600 dpm divided by 84 cpm=1,198 discharges/count (photons/count), which is used as a conversion factor to calculate the number of photons emitted by plant and detected by the camera Using the above conditions and calculation, various wild type and transformed plants were examined and photons/mm²/sec were determined. No visibly detectable bioluminescence was observed in the Columbia wt plants, the 35S LUC-transformed plants or Supernova plants, whereas all of the TK1829 transformed plants were visibly bioluminescent. As shown in Table 4, a threshold value of visible bioluminescence occurs between about 700,000 photons/mm²/sec and 1×10⁶ photons/mm²/sec (compare Tables 4c and 4d; corresponding to 126.4 and 1562.4 pg luc⁺/μg protein, respectively, as determined by luminometry and spectrophotometry; see Example 2B).

TABLE 4

| | | Photon Emission |
|---|---|---|
| Plant Comparison | | (cts/mm²/sec) |
| a) | Columbia wild-type | 16.8 |
| b) | 35S:Luc | 10,063.0 |
| c) | Supernova | 701,669.0 |
| d) | Tk1829-19-transformed Columbia | 1,061,668.0 |
| e) | TK1829-11-transformed Supernova | 7,160,446.0 |
| f) | TK1829-11-transformed Supernova | 52,604,180.0 |
| | with Norflurazon | |

These results demonstrate that the amount of light produced by the various transformed plants correlates with the amount of luciferase expressed in the plants, and establish standard conditions defining a threshold level of light emission required for visible bioluminescence.

These results further provide guidelines for generating other constructs, which contain various combinations of regulatory elements, that can be examined for the ability to confer visible bioluminescence on a plant. For example, by comparing a plant transformed with a recombinant nucleic acid molecule comprising a nucleotide sequence encoding a bioluminescent polypeptide operatively linked to one or more regulatory elements, with plants transformed with a 35S:Luc construct or a cab2:Luc construct, recombinant nucleic acid molecules encompassed within the present invention and useful for generating visibly bioluminescent plant cells or transgenic plants comprising such cells can be obtained. More directly, by transforming plants with various constructs and determining photon emission under standardized conditions as described above, visibly bioluminescent plants can be obtained.

Furthermore, the results disclosed herein indicate that plant cells other than Arabidopsis plant cells can be genetically modified using, for example, a TK1829 recombinant nucleic acid molecule, thus providing a means to obtain different species, strains and varieties of visibly bioluminescent transgenic plants. In this respect, genetically modified petunia plants that exhibit exceptional bioluminescence also have been generated using the disclosed compositions and methods.

D. Preparation and Characterization of Bioluminescent Petunia Plants

pMT-OM-LUC⁺ or pACT-OM-LUC⁺ expression constructs were introduced from MT-OM-LUC⁺ or pACT-OM-LUC⁺ into Agrobacterium strain ABI 1 and in planta transformation of Arabidopsis and of petunia plants were performed as described above.

Whole leaves from 10 week old petunia plants were removed and washed in warm soapy water. Leaves were sterilized in 10% (v/v)chlorine for 15 min and rinsed 4 times with sterile water. Squares of approximately 1 cm were cut from the leaves under sterile conditions with an scalpel and cultured for one day in pre-culture medium (MS medium supplemented with 1 mg/l of 6-benzylaminopurine (BAP) and 0.1 μg/ml of alpha-naphthalene acetic acid (NAA).

A. tumefaciens strain ABI carrying one of the plasmids was used to inoculate the pieces of leaves. Bacteria from a single colony were grown at 28° C. overnight in liquid LB medium on a rotary shaker (250 rpm). The medium was supplemented with 50 μg/ml kanamycin, 100 μg/ml spectinomycin and 34 μg/ml chloramphenicol. The next day, a 1:10 dilution was made into LB medium (without antibiotics) and sub-cultured cells were grown for 4 to 5 hr. Cultures were diluted to an OD (660 nm) of 0.05 to 0.07 with pre-culture liquid medium.

Explants were inoculated with a log phase solution of Agrobacterium adjusted to approximately 5×10⁸ cells. The leaf pieces were allow to soak for 5 min in the bacteria solution, blotted on sterile Whatman filter discs to remove excess of solution, then replaced in the original pre-culture plates and incubated for 2 days at 23° C. under low light. The leaf squares were transferred into shoot proliferation medium (pre-culture medium containing 75 μg/ml gentamycin and 500 μg/ml carbenicillin). After 4 weeks, shoots were excised and placed on rooting medium (MS medium supplemented with 60 μg/ml gentamycin and 500 μg/ml carbenicillin). After approximately 5 weeks, the plants were transferred to soil.

As shown in Table 5, Arabidopsis plants containing the MT-OM-LUC⁺ construct (designated “31” in Table 5) or the ACT-OM-LUC⁺ construct (“#27” and “#17”) were visibly biolunminescent (i.e., greater than 750,000 photons of light/mm²/sec (column labeled “photons”). These results demonstrate that the methods of the invention can be used to make various constructs, which, when introduced into plant cells, result in the generation of visibly bioluminescent plants.

Similarly, introduction of the TK 1829 construct or the ACT-OM-LUC⁺ construct into petunia plants resulted in the generation of bioluminescent petunia plants. Although petunia plants containing the Mr-OM-LUC⁺ construct were not examined for photon counts, the mean RLU/μg was similar to those for the TK 1828 and ACT-OM-LUC⁺ transformed petunias and the plants were visibly bioluminescent to the eye.

These results demonstrate that the constructs of the invention are useful for generating a variety of visibly bioluminescent plants. Together, the results of these studies demonstrate the broad utility of the claimed methods for generating visibly bioluminescent plants.

Although the invention has been described with reference to the above examples, it will be understood that modifications and variations are encompassed within the spirit and scope of the invention. Accordingly, the invention is limited only by the following claims.

TABLE 5

| 21 day old | | Weight | OD | OD | conc. | | | mean | pgLuc/ | | |
|---|---|---|---|---|---|---|---|---|---|---|---|
| plants | | ug | (750 nm) | (750 nm) | (ug/ul) | RLU 1 | RLU 2 | RLU/ug | ug | cts/mm²/sec | photons |
| | MT Line | | | | | | | | | | |
| 31902 | 31 | 99.3 | 0.086 | 0.086 | 0.0238844 | 128.1 | 132.8 | 1092.34395 | 15.24 | 2408.4 | 2,885,263.20 |
| | Actin Lines | | | | | | | | | | |
| | Actin 2-7 Col | | | | | | | | | | |
| 31902 | #27 | 98.9 | 0.047 | 0.046 | 0.0111278 | 38.37 | 39.6 | 700.68 | 9.80 | 911.64 | 1,092,144.72 |
| | Actin 1-1 Col | | | | | | | | | | |
| 31902 | #17 | 98.2 | 0.109 | 0.109 | 0.0313123 | 318.4 | 331.5 | 2,075.54 | 28.81 | 3342.4 | 4,004,195.20 |
| | Petunia | | | | | | | | | | |
| | Actin Line T3 | 102 | 0.062 | 0.065 | 5.0861595 | 31.46 | 30.96 | 1.23 | 0.02 | 840 | 1,006,320.00 |
| | TK1829 Line T5 | 96 | 0.105 | 101 | 84.933336 | 525.9 | 520.1 | 1.23 | 0.02 | 3385.7 | 4,056,068.60 |
| | MT Line T3 | 90 | 0.106 | 0.105 | 3.6981111 | 22.82 | 20.23 | 1.16 | 0.02 | n/a | |
| | control | 27 | 0.089 | 0.09 | 0.0150032 | 0.028 | 0.021 | 0.33 | 0.00 | 16.35 | 19,587.30 |

1. A genetically modified plant cell, comprising a heterologous nucleotide sequence encoding a bioluminescent polypeptide, wherein the bioluminescent polypeptide can be expressed in amount sufficient to produce at least 750,000 photons of visible light/mm²/second.

2. The genetically modified plant cell of claim 1, wherein the bioluminescent polypeptide is luciferase.

3. The genetically modified plant cell of claim 1, wherein the bioluminescent polypeptide is a luciferase variant.

4. The genetically modified plant cell of claim 3, wherein the luciferase variant is a luc⁺ luciferase variant.

5. The genetically modified plant cell of claim 4, wherein the luc⁺ luciferase variant is expressed in the plant cell at a level of at least 100 pg/μg protein as determined by western blot analysis.

6. The genetically modified plant cell of claim 1, wherein the bioluminescent polypeptide can be expressed in amount sufficient to produce at least one million photons of visible light/mm²/second.

7. The genetically modified plant cell of claim 1, which has a reduced level of chlorophyll as compared to corresponding wild type plant cell.

8. A transgenic plant, comprising the genetically modified plant cell of claim 1 or claim 7.

9. A plant cell or tissue obtained from the transgenic plant of claim 8.

10. A cutting of the transgenic plant of claim 8.

11. A seed produced by the transgenic plant of claim 8.

12. A cDNA or genomic DNA library prepared from the transgenic plant of claim 8, or from a plant cell or plant tissue obtained from said transgenic plant.

13. The transgenic plant of claim 8, wherein the plant is a monocot.

14. The transgenic plant of claim 8, wherein the plant is a dicot.

15. The transgenic plant of claim 14, wherein the plant is an angiosperm.

16. The transgenic plant of claim 15, wherein the angiosperm is a cereal plant, a leguminous plant, an oilseed plant, or a hardwood tree.

17. The transgenic plant of claim 8, wherein the plant is an ornamental plant.

18. The transgenic plant of claim 17, wherein the ornamental plant is a petunia.

19. The transgenic plant of claim 17, wherein the ornamental plant is a carnation.

20. The genetically modified plant of claim 1, wherein the heterologous nucleotide sequence encoding a bioluminescent polypeptide comprises nucleotides 8366 to 11,113 of SEQ ID NO:2.

21. The genetically modified plant of claim 1, wherein the heterologous nucleotide sequence encoding a bioluminescent polypeptide comprises nucleotides 8340 to 12,098 of SEQ ID NO:4 or nucleotides 6 to 3570 of SEQ ID NO:5.

22. A recombinant nucleic acid molecule, comprising, in operative linkage, a plant translational enhancer and a nucleotide sequence encoding a bioluminescent polypeptide.

23. The recombinant nucleic acid molecule of claim 22, wherein the plant translational enhancer is a plant potyvirus translational enhancer.

24. The recombinant nucleic acid molecule of claim 23, wherein the plant potyvirus is a tobacco etch virus (TEV) or an omega enhancer.

25. The recombinant nucleic acid molecule of claim 22, wherein the bioluminescent polypeptide is a luciferase polypeptide or variant thereof.

26. The recombinant nucleic acid molecule of claim 22, further comprising an operatively linked transcriptional regulatory element, which comprises a promoter.

27. The recombinant nucleic acid molecule of claim 26, wherein the transcriptional regulatory element is a cauliflower mosaic virus (CaMV) 35S regulatory element, a metallothionein regulatory element, or an actin 2 regulatory element.

28. The recombinant nucleic acid molecule of claim 22, comprising, in operative linkage, a CaMV 35S enhancer, a CaMV 35S promoter, a TEV translational enhancer, a nucleotide sequence encoding a luciferase polypeptide or variant thereof, and a CaMV 35S terminator.

29. The recombinant nucleic acid molecule of claim 28, wherein the CaMV 35S enhancer is a dual CaMV 35S enhancer.

30. The recombinant nucleic acid molecule of claim 28, wherein the luciferase polypeptide or variant thereof is a luc⁺ polypeptide.

31. The recombinant nucleic acid molecule of claim 22, comprising, in operative linkage, a metallothionein gene transcriptional regulatory element, an omega translational enhancer, a nucleotide sequence encoding a luciferase polypeptide or variant thereof, and an RbcS E9 polyA sequence.

32. The recombinant nucleic acid molecule of claim 22, comprising, in operative linkage, an actin 2 gene transcriptional regulatory element, an omega translational enhancer, a nucleotide sequence encoding a luciferase polypeptide or variant thereof, and an RbcS E9 polyA sequence.

33. The recombinant nucleic acid molecule of claim 22, comprising nucleotides 8366 to 11,113 of SEQ ID NO:2.

34. The recombinant nucleic acid molecule of claim 22, comprising nucleotides 8340 to 12,098 of SEQ ID NO:4 or nucleotides 6 to 3570 of SEQ ID NO:5.

35. A vector, comprising the recombinant nucleic acid molecule of claim 22.

36. The vector of claim 32, wherein the vector is an expression vector.

37. The vector of claim 35, wherein the recombinant nucleic acid molecule comprises, in operative linkage, a CaMV 35S enhancer, a CaMV 35S promoter, a TEV translational enhancer, a nucleotide sequence encoding a luciferase polypeptide or variant thereof, and a CaMV 35S terminator.

38. The vector of claim 35, wherein the recombinant nucleic acid molecule comprises, in operative linkage, a metallothionein gene transcriptional regulatory element, an omega translational enhancer, a nucleotide sequence encoding a luciferase polypeptide or variant thereof, and an RbcS E9 polyA sequence.

39. The vector of claim 35, wherein the recombinant nucleic acid molecule comprises, in operative linkage, an actin 2 gene transcriptional regulatory element, an omega translational enhancer, a nucleotide sequence encoding a luciferase polypeptide or variant thereof, and an RbcS E9 polyA sequence.

40. The vector of claim 35, wherein the recombinant nucleic acid molecule comprises nucleotides 8366 to 11,113 of SEQ ID NO:2.

41. The vector of claim 35, wherein the recombinant nucleic acid molecule comprises nucleotides 8340 to 12, 098 of SEQ ID NO:4 or nucleotides 6 to 3570 of SEQ ID NO:5.

42. The vector of claim 35, which is selected from SEQ ID NO:1 and SEQ ID NO:2.

43. The vector of claim 35, which is selected from SEQ ID NO:4 and SEQ ID NO:5.

44. A cell containing the recombinant nucleic acid molecule of claim 22.

45. A cell containing the vector of claim 35.

46. A method of producing a genetically modified plant cell that is visibly bioluminescent, the method comprising introducing a transgene comprising a nucleotide sequence encoding a bioluminescent polypeptide into a plant cell, whereby the bioluminescent polypeptide is expressed at a level that produces at least 750,000 photons of visible light/mm²/second, thereby producing a genetically modified plant cell that visibly bioluminesce.

47. The method of claim 45, wherein the transgene further comprises a plant translational enhancer operatively linked to the nucleotide sequence encoding a bioluminescent polypeptide.

48. The method of claim 46, wherein the plant translational enhancer is a plant potyvirus translational enhancer selected from a tobacco etch virus (TEV) translational enhancer and a tobacco mosaic virus translational enhancer.

49. The method of claim 46, wherein the plant translational enhancer is an omega translational enhancer having a nucleotide sequence set forth as nucleotides 1169 to 1235 of SEQ ID NO:5.

50. The method of claim 46, wherein the bioluminescent polypeptide is a luciferase polypeptide or variant thereof.

51. The method of claim 47, wherein the transgene further comprises a transcriptional regulatory element operatively linked to the plant translational enhancer and nucleotide sequence encoding a bioluminescent polypeptide.

52. The method of claim 51, wherein the transcriptional regulatory element is a cauliflower mosaic virus (CaMV) 35S regulatory element, a metallothionein gene regulatory element, or an actin gene regulatory element.

53. The method of claim 46, wherein the transgene comprises, in operative linkage, a dual CaMV 35S enhancer, a CaMV 35S promoter, a TEV translational enhancer, a nucleotide sequence encoding a luciferase polypeptide or variant thereof, and a CaMV 35S terminator.

54. The method of claim 53, wherein the luciferase polypeptide or variant thereof is a luc⁺ polypeptide.

55. The method of claim 46, wherein the transgene comprises, in -operative linkage, a metallothionein gene transcriptional regulatory element, which comprises a promoter and, optionally, an enhancer; an omega translational enhancer; a nucleotide sequence encoding a luciferase polypeptide or variant thereof; and an RbcS E9 polyA sequence.

56. The method of claim 55, wherein the luciferase polypeptide or variant thereof is a luc⁺ polypeptide.

57. The method of claim 46, wherein the transgene comprises, in operative linkage, an actin 2 gene transcriptional regulatory element, which comprises a promoter and, optionally, an enhancer; an omega translational enhancer; a nucleotide sequence encoding a luciferase polypeptide or variant thereof; and an RbcS E9 polyA sequence.

58. The method of claim 57, wherein the luciferase polypeptide or variant thereof is a luc⁺ polypeptide.

59. The method of claim 46, wherein the transgene comprises nucleotides 8366 to 11,113 of SEQ ID NO:2, nucleotides 8340 to 12,098 of SEQ ID NO:4 or nucleotides 6 to 3570 of SEQ ID NO:5.

60. The method of claim 46, wherein the transgene is stably maintained in the plant cell genome.

61. The method of claim 60, wherein the transgene is integrated into the plant cell genome.

62. The method of claim 46, further comprising contacting the plant cell, or the genetically modified plant cell derived therefrom, with an agent that inhibits carotenoid synthesis, thereby reducing the amount of chlorophyll in the plant cell.

63. The method of claim 62, wherein the agent is norfluorazon.

64. A genetically modified plant cell produced by the method of claim 46 or claim 62.

65. A transgenic plant comprising the genetically modified plant cell of claim 64.

66. A genetically modified plant cell, comprising a heterologous nucleotide, which comprises, in operative linkage, a CaMV 35S enhancer, a CaMV 35S promoter, a TEV translational enhancer, a nucleotide sequence encoding a luciferase polypeptide or variant thereof, and a CaMV 35S terminator wherein the luciferase polypeptide or variant thereof can be expressed in amount sufficient to produce at least 750,000 photons of visible light/mm²/second.

67. The genetically modified plant cell of claim 66, wherein the heterologous nucleotide sequence encoding a bioluminescent polypeptide comprises nucleotides 8366 to 11,113 of SEQ ID NO:2.

68. A transgenic plant, comprising the genetically modified plant cell of claim 66 or claim 67.

69. A plant cell or tissue obtained from the transgenic plant of claim 68.

70. A cutting of the transgenic plant of claim 68.

71. A seed produced by the transgenic plant of claim 68.

72. A cDNA or genomic DNA library prepared from the transgenic plant of claim 68, or from a plant cell or plant tissue obtained from said transgenic plant.

73. A genetically modified plant cell, comprising a heterologous nucleotide sequence, which comprises, in operative linkage, a metallothionein gene transcriptional regulatory element, an omega translational enhancer, a nucleotide sequence encoding a luciferase polypeptide or variant thereof, and an RbcS E9 polyA sequence., wherein the luciferase polypeptide or variant thereof can be expressed in amount sufficient to produce at least 750,000 photons of visible light/mm²/second.

74. The genetically modified plant cell of claim 73, wherein the heterologous nucleotide sequence comprises nucleotides 8340 to 12,098 of SEQ ID NO:4

75. A transgenic plant, comprising the genetically modified plant cell of claim 73 or claim 74.

76. A plant cell or tissue obtained from the transgenic plant of claim 75.

77. A cutting of the transgenic plant of claim 75.

78. A seed produced by the transgenic plant of claim 75.

79. A genetically modified plant cell, comprising a heterologous nucleotide sequence, which comprises, in operative linkage, an actin 2 gene transcriptional regulatory element, an omega translational enhancer, a nucleotide sequence encoding a luciferase polypeptide or variant thereof, and an RbcS E9 polyA sequence, wherein the luciferase polypeptide or variant thereof can be expressed in amount sufficient to produce at least 750,000 photons of visible light/mm²/second.

80. The genetically modified plant cell of claim 79, wherein the heterologous nucleotide sequence comprises nucleotides 6 to 3570 of SEQ ID NO:5.

81. A transgenic plant, comprising the genetically modified plant cell of claim 79 or claim 80.

82. A plant cell or tissue obtained from the transgenic plant of claim 81.

83. A cutting of the transgenic plant of claim 81.

84. A seed produced by the transgenic plant of claim 81.

85. A kit, comprising the genetically modified plant cell of claim 1 or claim 7, or a derivative of the genetically modified plant cell.

86. A kit comprising a derivative of the genetically modified plant cell, wherein the derivative of the genetically modified plant cell is a transgenic plant comprising the genetically modified plant cell of claim 1 or claim 7, or a cell, tissue or organ of said transgenic plant.

87. The kit of claim 86, wherein the organ of said transgenic plant is a flower or a bract.

88. The kit of claim 85, further comprising an amount of luciferin sufficient for generating visible bioluminescence of the genetically modified plant cell or the derivative of the genetically modified plant cell.

89. The kit of claim 85, further comprising an amount of a carotenoid inhibitor sufficient for reducing or inhibiting chlorophyll production in the genetically modified plant cell or the derivative of the genetically modified plant cell.

90. A kit, comprising a cutting or a seed of the transgenic plant of claim 8.

91. The kit of claim 90, further comprising reagents for growing a transgenic plant from the cutting or seed.

92. The kit of claim 90, further comprising an amount of a carotenoid inhibitor sufficient for reducing or inhibiting chlorophyll production in a transgenic plant grown from the cutting or seed.

93. The kit of claim 90, further comprising an amount of luciferin sufficient for generating visible bioluminescence of a transgenic plant grown from the cutting or seed.

94. A kit, comprising the genetically modified plant cell of claim 66, 67, 73, 74, 79, or 80, or a derivative of the genetically modified plant cell.

95. A kit comprising a derivative of a genetically modified plant cell, wherein the derivative of the genetically modified plant cell is a transgenic plant comprising the genetically modified plant cell of claim 66, 67, 73, 74, 79 or 80, or a cell, tissue or organ of said transgenic plant.

96. The kit of claim 95, wherein the organ of said transgenic plant is a flower or a bract.

97. The kit of claim 94, further comprising an amount of luciferin sufficient for generating visible bioluminescence of the genetically modified plant cell or the derivative of the genetically modified plant cell.

98. The kit of claim 94, further comprising an amount of a carotenoid inhibitor sufficient for reducing or inhibiting chlorophyll production in the genetically modified plant cell or the derivative of the genetically modified plant cell.

99. A kit, comprising a cutting of the transgenic plant of claim 68, or a seed produced by said transgenic plant.

100. The kit of claim 99, further comprising reagents for growing a transgenic plant from the cutting or seed.

101. The kit of claim 99, further comprising an amount of a carotenoid inhibitor sufficient for reducing or inhibiting chlorophyll production in a transgenic plant grown from the cutting or seed.

102. The kit of claim 99, further comprising an amount of luciferin sufficient for generating visible bioluminescence of a transgenic plant grown from the cutting or seed.

103. The kit of claim 95, further comprising an amount of luciferin sufficient for generating visible bioluminescence of the genetically modified plant cell or the derivative of the genetically modified plant cell.

104. The kit of claim 95, further comprising an amount of a carotenoid inhibitor sufficient for reducing or inhibiting chlorophyll production in the genetically modified plant cell or the derivative of the genetically modified plant cell.

105. A kit comprising a cutting of the transgenic plant of claim 75, or a seed produced by said transgenic plant.

106. The kit of claim 105, further comprising reagents for growing a transgenic plant from the cutting or seed.

107. The kit of claim 105, further comprising an amount of a carotenoid inhibitor sufficient for reducing or inhibiting chlorophyll production in a transgenic plant grown from the cutting or seed.

108. The kit of claim 105, further comprising an amount of luciferin sufficient for generating visible bioluminescence of a transgenic plant grown from the cutting or seed.

109. A kit comprising a cutting of the transgenic plant of claim 81, or a seed produced by said transgenic plant.

110. The kit of claim 109, further comprising reagents for growing a transgenic plant from the cutting or seed.

111. The kit of claim 110 further comprising an amount of a carotenoid inhibitor sufficient for reducing or inhibiting chlorophyll production in a transgenic plant grown from the cutting or seed.

112. The kit of claim 109, further comprising an amount of luciferin sufficient for generating visible bioluminescence of a transgenic plant grown from the cutting or seed.


SEQUENCE LISTING (5 sequences)

SEQ ID NO: 1 - DNA, 5475 bp, Artificial sequence
  Sequence for pTK1829 Vector
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtggtcccaaagatggacccccacccacgaggaacatcgtggaaaaagaagacgttccaaccacgtcttcaaagcaagtggattgatgtgataacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattcaacacaacatatacaaaacaaacgaatctcaagcaatcaagcattctacttctattgcagcaatttaaatcatttcttttaaagcaaaagcaattttctgaaaattttcaccatttacgaacgatagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtccgcaaatcaccagtctctctctacaaatctatctctctctattttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 2 - DNA, 11478 bp, Artificial sequence
  Sequence for pPZPTK1829 Vector
agtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctatagggactttaggtgatctggattttagtactggattttggttttaggaattagaaattttattgatagaagtattttacaaatacaaatacatactaagggtttcttatatgctcaacacgtgagcgaaaccctataagaaccctaattcccttatctgggaactactcacacattattatggagaaactcgatgtcgatcgactctagctagaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaccatggtcgatcgacagatctgcgaatcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttgcatgcctgcaggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcggactctagaattacacggcgatctttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgcgacacctgcgtcgaagatgttggggtgttggagcaagatggattccaattcagcgggagccacctgatagcctttgtacttaatcagagacttcaggcggtcaacgatgaagaagtgttcgtcttcgtcccagtaagctatgtctccagaatgtagccatccatccttgtcaatcaaggcgttggtcgcttccggattgtttacataaccggacataatcataggacctctcacacacagttcgcctctttgattaacgcccagcgttttcccggtatccagatccacaaccttcgcttcaaaaaatggaacaactttaccgaccgcgcccggtttatcatccccctcgggtgtaatcagaatagctgatgtagtctcagtgagcccatatccttgcctgatacctggcagatggaacctcttggcaaccgcttccccgacttccttagagaggggagcgccaccagaagcaatttcgtgtaaattagataaatcgtatttgtcaatcagagtgcttttggcgaagaaggagaatagggttggcaccagcagcgcactttgaatcttgtaatcctgaaggctcctcagaaacagctcttcttcaaatctatacattaagacgactcgaaatccacatatcaaatatccgagtgtagtaaacattccaaaaccgtgatggaatggaacaacacttaaaatcgcagtatccggaatgatttgattgccaaaaataggatctctggcatgcgagaatctcacgcaggcagttctatgaggcagagcgacacctttaggcagaccagtagatccagaggagttcatgatcagtgcaattgtcttgtccctatcgaaggactctggcacaaaatcgtattcattaaaaccgggaggtagatgagatgtgacgaacgtgtacatcgactgaaatccctggtaatccgttttagaatccatgataataattttttggatgattgggagctttttttgcacgttcaaaattttttgcaacccctttttggaaacgaacaccacggtaggctgcgaaatgcccatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtactcagcgtaagtgatgtccacctcgatatgtgcatctgtaaaagcaattgttccaggaaccagggcgtatctcttcatagccttatgcagttgctctccagcggttccatcttccagcggatagaatggcgccgggcctttctttatgtttttggcgtcttccatggctattaaaagaaatgatttaaatggttccaaagaagtagaaangntgatggcttaaattcgnttggtttgaaaaggttgggttaagattctcaagncctctccaaatgaaatgaacttccttanatanaggaagggtcttgcaaagaanaggggattgtgcgtcatccntacgnccagggnagatatcacatcaatccacttgctttgaagacgtgggttggaacgtcttcttttttccacgatgctcctcgtgggtggggntccatcttttgggaccactgtcggcagaggcatcttcaacgatggcctttcctttatcgcaatgatggcatttgtaggagccacttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaa

SEQ ID NO: 3 - DNA, 1216 bp, Artificial sequence
  Nucleotide sequence of the CaMV 35S promoter with duplicated enhancer, TEV leader and initial coding sequence, and CaMV 35S poly A signal sequence of pRTL2
aagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtcctcccaaagatggacccccacccacgaggaacatcgtggaaaaagaagacgttccaaccacgtcttcaaagcaagtggattgatgtgataacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatccactatccttcgcaagacccttcctctatataaggaagttcatttcatttggagaggacctcgagaattcaacacaacatatacaaaacaaacgaatctcaagcaatcaagcattctacttctattgcagcaatttaaatcatttcttttaaagcaaaagcaattttctgaaaattttcaccatttacgaacgatagccatggcactcatctttggcacagtcaacgctaacatcctgaaggaagtgttcggtggagctcggtacccggggatcctctagagtccgcaaatcaccagtctctctctacaaatctatctctctctattttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcaagctt

SEQ ID NO: 4 - DNA, 12497 bp, Artificial sequence
  MT-OM-LUC Vector
agtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctatagggactttaggtgatctggattttagtactggattttggttttaggaattagaaattttattgatagaagtattttacaaatacaaatacatactaagggtttcttatatgctcaacacgtgagcgaaaccctataagaaccctaattcccttatctgggaactactcacacattattatggagaaactcgatgtcgatcgactctagctagaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaccatggtcgatcgacagatctgcgaatcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtaccgtctgcagcgatgcatgttgtcaatcaattggcaagtcataaaatgcattaaaaaatattttcatactcaactacaaatccatgagtataactataattataaagcaatgattagaatctgacaaggattctggaaaattacataaaggaaagttcataaatgtctaaaacacaagaggacatacttgtattcagtaacatttgcagcttttctaggtctgaaaatatatttgttgcctagtgaataagcataatggtacaactacaagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcgaactcagtaggattctggtgtgtgcgcaatgaaactgatgcattgaacttgacgaacgttgtcgaaaccgatgatacgaacgaaagctctagctagaggatcaggcctgaattctctagaattacacggcgatctttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgcgacacctgcgtcgaagatgttggggtgttggagcaagatggattccaattcagcgggagccacctgatagcctttgtacttaatcagagacttcaggcggtcaacgatgaagaagtgttcgtcttcgtcccagtaagctatgtctccagaatgtagccatccatccttgtcaatcaaggcgttggtcgcttccggattgtttacataaccggacataatcataggacctctcacacacagttcgcctctttgattaacgcccagcgttttcccggtatccagatccacaaccttcgcttcaaaaaatggaacaactttaccgaccgcgcccggtttatcatccccctcgggtgtaatcagaatagctgatgtagtctcagtgagcccatatccttgcctgatacctggcagatggaacctcttggcaaccgcttccccgacttccttagagaggggagcgccaccagaagcaatttcgtgtaaattagataaatcgtatttgtcaatcagagtgcttttggcgaagaaggagaatagggttggcaccagcagcgcactttgaatcttgtaatcctgaaggctcctcagaaacagctcttcttcaaatctatacattaagacgactcgaaatccacatatcaaatatccgagtgtagtaaacattccaaaaccgtgatggaatggaacaacacttaaaatcgcagtatccggaatgatttgattgccaaaaataggatctctggcatgcgagaatctcacgcaggcagttctatgaggcagagcgacacctttaggcagaccagtagatccagaggagttcatgatcagtgcaattgtcttgtccctatcgaaggactctggcacaaaatcgtattcattaaaaccgggaggtagatgagatgtgacgaacgtgtacatcgactgaaatccctggtaatccgttttagaatccatgataataattttttggatgattgggagctttttttgcacgttcaaaattttttgcaacccctttttggaaacgaacaccacggtaggctgcgaaatgcccatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtactcagcgtaagtgatgtccacctcgatatgtgcatctgtaaaagcaattgttccaggaaccagggcgtatctcttcatagccttatgcagttgctctccagcggttccatcttccagcggatagaatggcgccgggcctttctttatgtttttggcgtcttccatggtcgactgtaattgtaaatagtaattgtaatgttgtttgttgtttgttgttgttggtaattgttgtaaaaataaagcttatcgatggatccgcccgtctagctcagttggtagagcgcaaggctcttaaccttgtggtcgtgggttcgagccccacggtgggcgatactttttatgcgattttgagatttgtgtcttaaaatgtaatcgtgataatattaactacgcaactgttcatttcaattcttgtgaacgtgtaaacctcgaaactacacaaatcaaatcacgagatcctttccttttttcatatagatactcttttacttttttatagaattagacttatcgacactccatgatgattcccatatggatggttctcatatttttaacattactttttacatagatcgaatttttggaaatgtatcgaaaaaagaggagagattcgattcaaatacccacatagaacggacaaggtcaataatgggcccactattcttttgagagcccatctcaacaagttgaatcatcttttttgtgtactctcacttcaccttttgacaaaaagtcatggaagccaaactttggaacactactttactttgccaacatcttgttttgttgaatacctttttcaaattggaaaccaaagacatatctaaagggaaaacctgtaggacaataaaacaaagaagtgatagctaaagtctaaactaatctcaatatatgtaaatcaaagagataactaatgaaaaatatgaaaattttcgcgaggattaatgaacaaaggaatttttttttttccttctaaaattttttattagacaattggtattacaatgacaactacgaagctaatacaaaacttcggaaaccaacttattacataagcaaaattatgtgtcatatactgaaaattacgaaactgaaaaagtgaatttaacgatttttcctttcgataagctttattcgctaacatgttaacatgttagaacatagaaaatattgaaaaaaaatttatgtttaaattgtcaaatggaaatatactttacacttttatttgtattaattgttactttgctctcctagttataaagaagtaaagtctatgattttgttacttctttttttcttgtaaatctttatacatgtaaaaagcttttgtttccaactctaatcctcaatattttatttttctgtttattggaaaagggctgtaaactaaaattatttacttgaggaacgattatttaggtgataagagtggacaaagatcgttgacacgtggacggtctacaaattctaattttgcctataaatatcaaagctcctgaatatgtaagtttcattcactgattatcgtttaaggcaaattaagatcatcttcataaatcttctcagatctcttccaattttctagaaaaaacggatcctctagagtcgacctgcaggcatgcaagctagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaa

SEQ ID NO: 5 - DNA, 12304 bp, Artificial sequence
  ACT-OM-LUC Vector
gatcccggatgtagtagtagccattatttaatgtacatactaatcgtgaatagtgatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaagttagatgaagtttgtgtatagattcttcgaaactttaggatttgtagtgtcgtacgttgaacagaaagctatttctgattcaatcagggtttatttgactgtattgaactctttttgtgtgtttgcagctcaggatccatcgataagctttatttttacaacaattaccaacaacaacaaacaacaaacaacattacaattactatttacaattacagtcgaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagaattcaggcctgatcctctagctagagctttcgttcgtatcatcggtttcgacaacgttcgtcaagttcaatgcatcagtttcattgcgcacacaccagaatcctactgagttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcgctgcagacggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgattcgcagatctgtcgatcgaccatggtacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttctagctagagtcgatcgacatcgagtttctccataataatgtgtgagtagttcccagataagggaattagggttcttatagggtttcgctcacgtgttgagcatataagaaacccttagtatgtatttgtatttgtaaaatacttctatcaataaaatttctaattcctaaaaccaaaatccagtactaaaatccagatcacctaaagtccctatagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctagcttgcatgcctgcaggtcgactctagag